SEQUENCE LISTING (6 sequences)

SEQ ID NO: 1 - DNA, 19 bp, Artificial Sequence
  Description of Artificial Sequence Synthetic oligonucleotide
ggagctggtgacgtaggca

SEQ ID NO: 2 - DNA, 19 bp, Artificial Sequence
  Description of Artificial Sequence Synthetic oligonucleotide
gcgcgctttgtaggattcg

SEQ ID NO: 3 - DNA, 46 bp, Artificial Sequence
  Description of Artificial Sequence Synthetic oligonucleotide
aaattagaaccaaatccagaaagcctggaactttcttttaccatgc

SEQ ID NO: 4 - DNA, 46 bp, Artificial Sequence
  Description of Artificial Sequence Synthetic oligonucleotide
gcatggtaaaagaaagttccaggctttctggatttggttctaattt

SEQ ID NO: 5 - DNA, 46 bp, Artificial Sequence
  Description of Artificial Sequence Synthetic oligonucleotide
gaaaaatggaacatgatggcagcctttttcaagcagtaggaattgg

SEQ ID NO: 6 - DNA, 46 bp, Artificial Sequence
  Description of Artificial Sequence Synthetic oligonucleotide
ccaattcctactgcttgaaaaaggctgccatcatgttccatttttc

(19) United States
(12) Patent Application Publication
(10) Pub. No.: US 2008/0306002 A1
(43) Pub. Date: Dec. 11, 2008

(54) Combination Therapy for Preventing Angiogenesis

(75) Inventors: Daniel C. Chung, Cambridge, MA (US); Yusuke Mizukami, Hokkaido (JP)

Correspondence Address: DAVID S. RESNICK, NIXON PEABODY LLP, 100 SUMMER STREET, BOSTON, MA, 02110-2131, US

(73) Assignee: The General Hospital Corporation, Boston, MA (US)

(21) Appl. No.: 12/063,071

(22) PCT Filed: Aug. 18, 2006
(86) PCT No.: PCT/US2006/032297

§ 371 Date: Jun. 20, 2008

Related U.S. Application Data
(60) Provisional application No. 60/709,156, filed on Aug. 18, 2005.

(51) Int. Cl. A61K 38/17; A61P 35/00

(52) U.S. Cl. 514/12

(57) ABSTRACT

The present invention is directed to a combination therapy for inhibiting angiogenesis in a subject having, or at risk of having, an angiogenic disease/disorder. The method comprises both administering to the mammal an inhibitor of HIF-I and administering a second compound or agent that inhibits angiogenesis such as an inhibitor of IL-8, VEGF, angiopoietins, EGF, FGF, TGF, G-CSF, or PDGF. Administration of an inhibitor of hypoxia inducible factor-1 (HIF-I) in combination with an inhibitor of interleukin 8 (IL-8) is particularly useful for treatment of colon cancer, lung cancer, pancreatic cancer, and breast cancer.

### CROSS REFERENCE TO RELATED APPLICATIONS

This Application claims the benefit under 35 U.S.C §119(e) of U.S. Provisional Application No. 60/709,156 filed Aug. 18, 2005.

### GOVERNMENT SUPPORT

This work was supported by the National Institute of Health Research Grant, NCI CA92594. The government has certain rights to this invention.

### FIELD OF INVENTION

The present invention relates to methods for inhibiting angiogenesis and to methods for treatment of cancer or diseases/disorders involving angiogenesis using a combination therapy that targets hypoxia inducible factor-1 (HIF-1) and an additional factor involved in angiogenesis, such as IL-8, VEGF, angiopoietins, EGF, FGF, TGF, G-CSF, or PDGF.

### BACKGROUND OF THE INVENTION

Angiogenesis or “neovascularization” is a multi-step process controlled by the balance of pro- and anti-angiogenic factors. The latter stages of this process involve proliferation and the organization of endothelial cells into tube-like structures. Growth factors such as fibroblast growth factor 2 (FGF2) and vascular endothelial growth factor (VEGF) are key players in promoting endothelial cell growth and differentiation and are involved in angiogenesis. Endothelial cells also respond to many cytokines during the angiogenic process.

Pathological neovascularization occurs in a variety of diseases such as psoriasis, haemangioblastoma, opthalmic and rheumatic diseases, solid tumor growth and ischemic diseases, for example caused by diabetes, coronary heart disease, or stroke. Ischemia ultimately leads to tissue hypoxia and the body's compensatory effect is to increase neovascularization.

Hypoxia inducible factor-1 (HIF-1) is a transcription factor that is considered a critical mediator of the cellular response to hypoxia through its regulation of genes that control angiogenesis¹⁻⁴. HIFs regulate the transcription of hypoxia-inducible genes by binding hypoxia response elements (HRE) found in the promoter and enhancer regions of inducible genes³². Hypoxia response elements have been found in the promoter regions of genes encoding VEGF, the VEGF receptor Flt-1, nitric oxide synthases (associated with vasodilatation)³¹. It is believed that hypoxia induces upregulation of VEGF and VEGFR gene expression by mechanisms involving hypoxia-inducible factors (HIFs). HIFs may also indirectly increase expression of angiogenic factors such as angiopoietins, FGFs, and PDGF through secondary cascades of gene regulation³¹. Thus, HIFs represent an attractive therapeutic target^{5,6} for treatment of angiogenesis related diseases and cancer.

Therapies directed against VEGF or its receptors have shown mixed efficacy in cancer treatment. Recently, this modality has received validation in a large, Phase III clinical trial in metastatic colorectal cancer patients. A monoclonal antibody to VEGF, Avastin, plus chemotherapy resulted in a highly significant longer time to progression and greater survival than chemotherapy alone³³ and was FDA approved in 2004 and approved by the European Union in 2005.

Although progress has been made in the field of anti-angiogenic therapy, there are still many areas involving angiogenic treatment that need to be improved.

### SUMMARY

We have discovered that when using agents that target HIF-1, there are compensatory effects that occur which preserve the induction of angiogenesis. For example, such effects include, but are not limited to, the persistent expression of VEGF and induction of the pro-angiogenic cytokine IL-8. Accordingly, we found that one needs to target a second angiogenic target when targeting HIF-1. For example, targeting IL-8 as well as HIF-1. Neutralization of IL-8 in HIF-1 deficient tumors results in a dramatic inhibition of angiogenesis and tumor growth. Furthermore, an additive effect on the inhibition of angiogenesis is observed when both IL-8 and VEGF are inhibited in HIF-1 deficient tumors.

In addition, we have found that when the expression of HIF-1 is transiently inhibited in cancer cell lines, hypoxic induction of the angiogenic cytokine IL-8 occurs only in a subset of cases. For example, upon inhibition of HIF-1, significant induction of IL-8 occurs in colon cancer, lung cancer, pancreatic cancer, and breast cancer cell lines, while there is an absence of induction in gastric cancer cells, liver cancer cells, cervical cancer cells and prostate cancer cells. Thus a combination therapy targeting both IL-8 and HIF-1 is particularly relevant for treatment of colon cancer, lung cancer, pancreatic cancer, and breast cancer.

Accordingly, the present invention is directed to a combination therapy for treating disorders caused by undesired angiogenesis in a tissue of a subject having, or at risk of having, an angiogenic disease/disorder. The treatment comprises administering to the subject both an inhibitor of HIF-1 and a second compound or agent that inhibits angiogenesis.

The compounds/agents for use in the combination therapy can be administered to the patient simultaneously. Alternatively, the compounds/agents can be administered sequentially. This can range from hours, days or weeks, e.g. 14 days, of each other.

In one embodiment, the combination therapy comprises administering to a subject an inhibitor of HIF-1 and an inhibitor of interleukin 8 (IL-8).

In one embodiment, the combination therapy comprises administering to a subject an inhibitor of HIF-1 and an inhibitor of a growth factor involved in angiogenesis, e.g. an inhibitor of vascular endothelial growth factor (VEGF), platelet derived growth factor (PDGF), fibroblast growth factor (FGF), transforming growth factor (TGF), epidermal growth factor (EGF), or granulocyte colony stimulating factor (GCSF), or a direct angiogenesis inhibitor such as endostatin, angiostatin, thrombospondin, or tumstatin.

In one embodiment, HIF-1 and an inhibitor of VEGF is administered.

In one embodiment, HIF-1 and an inhibitor of PDGF is administered (e.g PDGF A or PDGF B).

In one embodiment, the combination therapy comprises administering to a subject an inhibitor of HIF-1 and an inhibitor of angiopoietin 1.

The combination therapy of the invention can be administered to inhibit angiogenesis in a subject that has, or that is at risk of having any angiogenic disease or disorder. Angiogenesis plays a role in a variety of disease processes. By inhibiting angiogenesis, one can intervene in the disease, ameliorate the symptoms, and in some cases cure the disease.

In one embodiment, the angiogenic disease or disorder to be treated in the subject is cancer. The combination therapy can be directed to the treatment of a solid tumor or solid tumor metastasis. Alternatively, the combination therapy can be directed to the treatment of a blood borne or bone marrow derived tumors such as leukemia, multiple myeloma or lymphoma.

In one embodiment, the combination therapy is directed to the treatment of retinal tissue and said disease or disorder is retinopathy, diabetic retinopathy, or macular degeneration.

In one embodiment, the methods of the present invention are directed toward treatment of atherosclerosis or a tissue at risk of restenosis, wherein the tissue is at the site of coronary angioplasty.

In one embodiment, the combination therapy of the invention is directed toward treating undesired angiogenesis in a subject, such as in a tissue wherein the tissue is inflamed and said disease or disorder is arthritis (rheumatoid or osteo-arthritis).

Also provided is a method of treating cancer using a combination therapy comprising i) administering to said subject an inhibitor of hypoxia inducible factor-1 (HIF-1) and ii) administering to said subject an inhibitor of interleukin 8 (IL-8), wherein the cancer is selected from the group consisting of colon cancer, pancreatic cancer, lung cancer and breast cancer.

The methods of the present invention can be used either alone, or in conjunction with other treatment methods known to those of skill in the art. For example, such methods may include, but are not limited to, chemotherapy, radiation therapy, or surgery.

In one embodiment, the combination therapy that comprises administering to the subject an inhibitor of HIF-1 and administering a second compound or agent that inhibits angiogenesis further comprises administration of a third agent or compound that inhibits angiogenesis.

In one embodiment, the combination therapy comprises administering to a subject an inhibitor of HIF-1, administering an inhibitor of IL-8, and administering an inhibitor of VEGF.

In one embodiment, the combination therapy comprises administering to a subject an inhibitor of HIF-1, administering an inhibitor of IL-8, and administering an inhibitor of PDGF.

Administration of the inhibitors can be performed by intravenous, intramuscular, subcutaneous, intradermal, topical, intraperitoneal, intrathecal, intrapleural, intrauterine, rectal, vaginal, intrasynovial, intraocular/periocular, intratumor or parenteral administration.

In one embodiment, the subject is at risk for developing said angiogenic disease or disorder and the combination therapy is administered prophylactically. The risk can be determined genetically. Alternatively, the risk can be determined by measuring levels of marker proteins in the biological fluids (i.e. blood, urine) of a patient. For example, cancer marker proteins include markers such as calcitonin, PSA, thymosin β-15, thymosin β-16, and matrix metalloproteinases (MMPs).

The invention further provides for kits comprising an inhibitor of hypoxia inducible factor-1 (HIF-1) and at least one other anti-angiogenic agent. The kits are designed to be used with a HIF-1 inhibitor, thus a second anti-angiogenic agent is specifically selected to compensate for properties exhibited by inhibition of HIF-1, such as the induction of IL-8, which is a pro-angiogenic factor. Additionally, inhibition of HIF-1 does not completely stop the expression of VEGF, thus it is preferred that the kit contain at least an IL-8 inhibitor, or a VEGF inhibitor, preferably an IL-8 inhibitor. In this way targeting both IL-8 and HIF-1 compensates for the undesirable induction of IL-8 by inhibition of HIF-1.

In one embodiment, a kit designed for treatment of a subject having, or at risk of having, an angiogenic disease or disorder is provided that contains an inhibitor of hypoxia inducible factor-1 (HIF-1) and at least one other anti-angiogenic agent that is a direct inhibitor of angiogenesis, e.g. endostatin, angiostatin, thrombospondin and tumstatin.

### BRIEF DESCRIPTION OF FIGURES

FIGS. 1A to 1E show the growth of DLD-1^{HIF-kd} cells in vivo. (FIG. 1A) Tumor volume and weight of DLD-1^{HIF-wt} and DLD-1^{HIF-kd} xenografts. * indicates P<0.05. (FIG. 1B) Imnunoblotting for HIF-1α and Glut-1 in DLD-1^{HIF-kd} xenografts. VEGF mRNA and protein levels in cultured DLD-1 cells (FIG. 1C) and in tumor xenografts (FIG. 1D) were measured. (FIG. 1E) Immunohistochemistry for CD-31/PECAM and quantification of microvessel density in DLD-1 xenografts (bar=100 μm).

FIGS. 2A to 2F show that knock-down of HIF-1 facilitates the induction of IL-8 by NF-κB during hypoxic conditions. IL-8 mRNA and protein levels in (FIG. 2A) cultured DLD-1 cells and (FIG. 2B) DLD-1 xenografts. (FIG. 2C) IL8 promoter activity during hypoxia in DLD-1^{HIF-kd} and DLD-1^{HIF-wt} cells. (FIG. 2D) NF-κB reporter activity in hypoxic conditions in DLD-1^{HIF-kd} cells. In (B,C,D), open bars=normoxic conditions, solid bars=hypoxic conditions. (FIG. 2E) Immunoblotting for NF-κB, p65 subunit and phospho-p65^{Ser536} (p-p65), in DLD-1 tumor lysates. (FIG. 2F) Effect of NF-κB inhibition on IL-8 promoter activity with BAY 11-7082.

FIGS. 3A to 3H show the increased production of reactive oxygen species in HIF-kd cells expressing K-ras. (FIG. 3A) Increased production of hydrogen peroxide in DLD-1^{HIF-kd} cells as measured by Amplex Red (left panel) and DCF fluorescence (right panel). (FIG. 3B) Effect of inhibitors of hydrogen peroxide production, N-Acetyl-L-cysteine (NAC), pryrolidinedithiocarbamate (PDTC), rotenone (Rot), and diphenylene iodonium (DPI) on induction of NF-κB reporter activity by hypoxia. * P<0.01. (FIG. 3C) Induction of IL8 gene expression by t-butyl hydroperoxide (t-BH) and inhibition by 5 μM BAY 11-7082. (FIG. 3D) Synergistic effect of hypoxia and KRAS^{V12} on IL-8 induction in Caco2^{HIF-kd} cells. (FIG. 3E) Induction of IL-8 promoter activity by KRAS^{V12} and hypoxia and inhibition by BAY 11-7082. Open bars=normoxia, solid bars=hypoxia. (FIG. 3F) NF-κB reporter activity (left panel) and IL-8 promoter activity (right panel) after silencing of endogenous mutant KRAS by siRNA, pSR/K-ras^{D13}. (FIG. 3G) IL8 mRNA levels in DLD-1^{HIF-kd} cells after silencing of endogenous mutant KRAS by pSR.K-ras^{D13}. Open bars=normoxia, solid bars=hypoxia. (FIG. 3H) Effect of KRAS^{V12} and 40 μM t-butyl hydroperoxide (t-BH) on NF-κB reporter activity in Caco2 cells.

FIGS. 4A to 4F show the role of IL-8 in tumor angiogenesis in vivo. Tumor volume (FIG. 4A) and weight (FIG. 4B) of DLD-1^{HIF-wt} and DLD-1^{HIF-kd} xenografts after treatment with neutralizing antibody to IL-8, MAB208 (* P<0.01, DLD-1^{HIF-kd}+IgG vs. DLD-1^{HIF-kd}+MAB208). There was no change in the percentage of non-necrotic viable tumor with MAB208 treatment (DLD-1^{HIF-wt}:69.8% vs. 69.9%; DLD-1^{HIF-kd}:87.8% vs. 83.9%, IgG vs. MAB208, P NS). (FIG. 4C) Ki-67 labeling and TUNEL indices in MAB208 treated xenografts. (FIG. 4D) Growth of DLD-1 cells in the presence of MAB208 under hypoxic conditions. (FIG. 4E) Blood vessels were visualized by CD31 immunohistochemistry and lectin perfusion and microvessel density graphed (number of vessels per field. (FIG. 4F) Growth of DLD-1^{HIF-kd} xenografts when treated with a neutralizing VEGF antibody (MAB293) and/or a neutralizing antibody to IL-8 (MAB208). In these xenografts, the percentage of viable non-necrotic tumor fell slightly to 74.7% from 87.8% in animals who received control antibody only (P=0.1).

FIGS. 5A and 5B show graphs depicting the growth of Caco2^{HIF-wt} and Caco2^{HIF-kd} cells in vitro (FIG. 5A) and in vivo as xenografts in nude mice (FIG. 5B). The growth curves of the cells in vitro are illustrated as the fold increase compared to baseline.

FIG. 6 shows an immunoblot of HIF-1α and HIF-2α proteins in DLD-^{1HIF-WT, DLD-}1^{HIF-kd/1470}, and DLD-1^{HIF-kd/2192} cells in normoxic (N) and hypoxic (H) conditions. No HIF-1α is detectable in the knock-down cells. HIF-2α protein levels are nearly undetectable in the parental DLD-1 cells, and there is no induction of HIF-2α in the HIF-1-kd cell lines in hypoxia. Similarly, DNA microarray studies indicated that mRNA levels for HIF-1α fell from 1518.2 relative units in DLD-1^{HIF-WT} cells to 124.5 relative units in DLD-1^{HIF-kd} cells in hypoxic conditions. HIF-2α mRNA levels, in contrast, were extremely low in DLD-1^{HIFWT} cells (54.5 relative units), and furthermore, there was no increase when HIF-1α was knocked-down in DLD-1^{HIF-kd} cells (43.2 relative units).

FIG. 7 shows a graph illustrating hypoxic induction of IL-8 in the absence of HIF-1α is observed in many cancer cell lines. HIF-1α was transiently knocked-down with two independent siRNA constructs (pSR.HIF-1α1470 and pSR.HIF-1α2192), and hypoxic induction of the IL-8 promoter was measured. Induction of IL-8 was observed in DLD-1, ColoHSR, SW480, and HCT116 colon cancer cells. Among the non-colonic cell lines tested, strong induction was also observed in pancreatic cancer cells (Panc-1, Capan-1), breast cancer cells (MDA-MB 453), and lung cancer cells (HOP-92), but not in gastric cancer cells (AGS), hepatoma cells (HepG2, HuH7), cervical cancer cells (HeLa), or prostate cancer cells (PC3). (* indicates p<0.05).

FIG. 8 shows the specificity of the pSR.^{HIF-1α1470} and pSR.^{HIF-1α2192} constructs. Specificity was demonstrated by co-expression of HIF-1α expression vectors with synonymous codon mutations that are not affected by the siRNA target sequences. Expression of the HIF-1αSDM1470 construct but not the HIF-1αSDM2192 construct strongly induced HRE reporter activity in DLD-1^{HIF-kd/1470} cells, and the hypoxic induction of IL-8 promoter activity seen in these cells was blocked. Similar results were obtained for the HIF-1αSDM2192 construct in DLD-1^{HIF-kd/2192} cells.

FIGS. 9A to 9B show graphs illustrating that oncogenic K-ras plays a role in the hypoxic induction of IL-8 in noncolonic cancers. (FIG. 9A); the pancreatic cancer cell line Panc-1 carries a mutant K-rasD12 oncogene. Knock-down of HIF-1α resulted in the induction IL-8 mRNA expression in these cells (top left panel). Immunoblotting demonstrated successful knock-down of HIF-1α protein (top right panel). When siRNA to K-rasD12 was also introduced, the hypoxic induction of IL-8 promoter activity was blunted (lower panel). (FIG. 9b); In PC3 prostate cancer cells that carry a wild type K-ras gene, introduction of oncogenic K-rasV12 in combination with transient knock-down of HIF-1α resulted in the hypoxic induction of the IL-8 promoter.

FIG. 10 shows a table illustrating the results of a cDNA microarray analysis that identified genes which were up-regulated at least 2-fold by hypoxia but whose expression was attenuated less than 30% when HIF-1 was silenced.

### DETAILED DESCRIPTION

The present invention relates generally to a method of treating undesired angiogenesis in a subject having, or at risk of having, an angiogenic disease or disorder, such as cancer. The methods of the invention are directed to a combination therapy wherein one component is an inhibitor of HIF-1, and the component is administered together with an effective amount of a second compound or agent having anti-angiogenic activity.

As used herein, the term “subject” or “patient” or refers to any mammal. The patient is preferably a human, but can also be a mammal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

The invention provides for a method for the inhibition of angiogenesis in a tissue of a subject, thereby disrupting events in the tissue which depend upon angiogenesis.

Angiogenesis plays a role in a variety of disease processes. By acting to disrupt undesired angiogenesis in a tissue, one can intervene in the disease, ameliorate the symptoms, and in some cases cure the disease. Where the growth of new blood vessels is the cause of, or contributes to, the pathology associated with a disease, blocking this undesired angiogenesis will reduce the deleterious effects of the disease. Examples include reduction in the systems of rheumatoid arthritis, obesity, diabetic retinopathy, inflammatory diseases, restenosis, and the like. Where the growth of new blood vessels is required to support growth of a deleterious tissue, disrupting angiogenesis will reduce the blood supply to the tissue and thereby contribute to reduction in tissue mass based on blood supply requirements. Examples include reduction in growth of tumors where neovascularization is a continual requirement in order that the tumor grows beyond a few millimeters in thickness, and for the establishment of solid tumor metastases.

Angiogenic diseases amenable to treatment with the present invention include but are not limited to diabetic retinopathy, macular degeneration, retrolental fibroplasia, trachoma, neovascular glaucoma, psoriases, angio-fibromas, immune and non-immune inflammation such as rheumatoid arthritis, capillary formation within atherosclerotic plaques, hemangiomas, excessive wound repair, and the like.

The combination therapy described herein is particularly useful in methods of treating angiogenesis at a site of tumorigenesis in a subject. Administering the combined therapy to such sites prevents or inhibits blood vessel formation thereby inhibiting the development and growth of the tumor. Tumors which may be treated by the combination therapy include but are not limited to melanoma, metastases, adenocarcinoma, sarcomas, thymoma, lymphoma, lung tumors, liver tumors, colon tumors, kidney tumors, non-Hodgkins lymphoma, Hodgkins lymphoma, leukemias, uterine tumors, breast tumors, prostate tumors, renal tumors, ovarian tumors, pancreatic tumors, brain tumors, testicular tumors, bone tumors, muscle tumors, tumors of the placenta, gastric tumors and the like.

The combination therapy described herein comprises the administration of a compound or agent that inhibits HIF-1 together with a compound or agent that disrupts angiogenesis, either simultaneously or sequentially.

As described herein, the compound or agent acting on blocking HIF-1 and the compound acting on a second site, can be DNA, RNA (e.g. anti-sense, siRNA, RNAi), a small organic molecule, a natural product, protein (e.g., antibody), peptide or peptidomimetic.

Any inhibitor of HIF-1 can be used in methods of the invention. As used herein, “inhibitor of HIF-1” means a compound or agent that inhibits the biological activity of HIF-1, interferes with the HIF-1 signal transduction pathway, or down regulates expression or availability of HIF-1 in a cell or organism. Many inhibitors of HIF-1 are known to those skilled in the art. For example, various inhibitors are described in PCT publications WO2004087066, WO2006023658, and WO2005046595, and U.S. patent applications 20050054720, 20050026872 and 20040087556, which are herein incorporated by reference. Other compounds that inhibit HIF-1 include, but are not limited to, PX-478, Panzem NCD (2-methoxyestradiol or 2ME2) (EntreMed, Inc., Rockville, Md.), and RX-0047. In addition, see Example 1 of this application for an example of inhibitory siRNAs that can be used in methods of the invention.

As used herein a “compound or agent that blocks or inhibits angiogenesis” or “anti-angiogenic compound or agent” is a compound or agent that is capable of inhibiting or reducing the formation of blood vessels.

Any compound or agent that inhibits angiogenesis can be used in the combination therapy method described herein. Methods for determining anti-angiogenic activity are well known to those skilled in the art, some of which are described within this application under the subheading, “Angiogenesis Screening Assays”.

Examples of angiogenesis inhibitors that can be used in methods of the invention include, but are not limited to, tyrosine kinase inhibitors, such as inhibitors of the tyrosine kinase receptors Flt-1 (VEGFR1) and Flk-1/KDR, inhibitors of epidermal-derived, fibroblast-derived, or platelet derived growth factors, MMP (matrix metalloprotease) inhibitors, integrin blockers, interferons, interleukin-12, pentosan polysulfate, cyclooxygenase inhibitors, including nonsteroidal anti-inflammatories (NSAIDs) like aspirin and ibuprofen as well as selective cyclooxygenase-2 inhibitors like rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), carboxyamidotriazole, combretastatin A-4, squalamine, 6-O-chloroacetyl-carbonyl) fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186) and inhibitors of the angiopoietins, such as angiopoietin 1. Antisense inhibitors of VEGF are described in PCT publication WO9739120.

Other known angiogenesis inhibitors include Bevacizumab (Avastin), Arresten, Canstatin, Combretastatin, Endostatin, NM-3, Thrombospondin, Tumstatin, 2-methoxyestradiol, Vitaxin, ZD1839 (Iressa), ZD6474, OSI774 (Tarceva), CI1033, PKI1666, IMC225 (Erbitux), PTK787, SU6668, SU11248, Herceptin, and IFN-α, CELEBREX® (Celecoxib), THALOMID® (Thalidomide), and IFN-α (Kerbel et al., Nature Reviews, Vol. 2, October 2002, pp. 727).

In one embodiment, the anti-angiogenesis inhibitor use in methods of the invention is an inhibitor of vascular endothelial derived growth factor (VEGF).

In another embodiment, the anti-angiogenesis inhibitor use in methods of the invention is an inhibitor of interleukin-8 (IL-8).

In one embodiment, both an inhibitor of VEGF and an inhibitor of IL-8 are administered to a subject together with a inhibitor of HIF-1.

In one preferred embodiment, the anti-angiogenesis inhibitor used in methods of the invention is an antibody. As used herein, the term “antibody”, includes human, humanized and animal mAbs, and preparations of polyclonal antibodies, as well as antibody fragments, synthetic antibodies, including recombinant antibodies (antisera), chimeric antibodies, including humanized antibodies, anti-idiotypic antibodies and derivatives thereof. Human and humanized antibodies are preferred.

Antibodies directed against various angiogenesis factors are well known to those skilled in the art. For example a neutralizing antibody against IL-8 has been commercially developed by Abgenix, Inc., Fremont, Calif. (Suyun Huang et al. Fully humanized neutralizing antibodies to interleukin-8 (ABX-IL8) inhibit angiogenesis, tumor growth, and metastasis of human melanoma American Journal of Pathology. 2002; 161:125-134.) Various humanized anti-IL-8 monoclonal antibodies are also described in U.S. Pat. No. 6,133,426. Various anti-IL-8 antibody fragment-polymer conjugates are described in U.S. Pat. No. 6,458,355.

Neutralizing antibodies that block signaling by VEGF receptors expressed on vascular endothelial cells to reduce tumor growth by blocking angiogenesis through an endothelial-dependent paracrine loop are disclosed in U.S. Pat. No. 6,365,157 and International Publications Nos. WO 00/44777, WO 01/54723, WO 01/74296, WO 01/90192, “Bispecific Antibodies That Bind to VEGF Receptors” (Zhu, International PCT application filed Jun. 26, 2002; WO 03/02144, WO 04/03211), and “Method of Treating Atherosclerosis and Other Inflammatory Diseases” (Carmeliet et al.; International PCT application filed Jun. 20, 2002; WO 03/00183).

In one preferred embodiment, an anti-VEGF monoclonal antibody is used as the agent that inhibits angiogenesis, e.g. Avastin™ (Genentech; South San Francisco, Calif.), which is a recombinant humanized antibody to VEGF. (See, WO 98/45331; WO 96/30046; and Middleton and Lapka, Clin J Oncol Nurs. 2004 December; 8(6):666-9, which are herein incorporated by reference).

Preferably the anti-VEGF monoclonal antibody is humanized (see for example WO 98/45331; WO 96/30046; and Kim et al., Growth Factors, 7:53-64 (1992)), the contents of each are herein incorporated by reference).

As used herein the term “VEGF inhibitors” refers to any compound or agent that produce a direct effect on the signaling pathways that promote growth, proliferation and survival of a cell by inhibiting the function of the VEGF protein, including inhibiting the function of VEGF receptor proteins. The term “agent” or “compound” as used herein means any organic or inorganic molecule, including modified and unmodified nucleic acids such as antisense nucleic acids, RNAi agents such as siRNA or shRNA, peptides, peptidomimetics, receptors, ligands, and antibodies. Preferred VEGF inhibitors, include for example, AVASTIN® described above (bevacizumab), an anti-VEGF monoclonal antibody of Genentech, Inc. of South San Francisco, Calif., VEGF Trap (Regeneron/Aventis). Additional VEGF inhibitors include CP-547,632 (3-(4-Bromo-2,6-difluoro-benzyloxy)-5-[3-(4-pyrrolidin 1-yl-butyl)-ureido]-isothiazole-4-carboxylic acid amide hydrochloride; Pfizer Inc., NY), AG13736, AG28262 (Pfizer Inc.), SU5416, SU11248, & SU6668 (formerly Sugen Inc., now Pfizer, New York, N.Y.), ZD-6474 (AstraZeneca), ZD4190 which iiibits VEGF-R2 and -R1 (AstraZeneca), CEP-7055 (Cephalon Inc., Frazer, Pa.), PKC 412 (Novartis), AEE788 (Novartis), AZD-2171), NEXAVAR® (BAY 43-9006, sorafenib; Bayer Pharmaceuticals and Onyx Pharmaceuticals), vatalanib (also known as PTK-787, ZK-222584: Novartis & Schering: AG), MACUGEN® (pegaptanib octasodium, NX-1838, EYE-001, Pfizer Inc./Gilead/Eyetech), IM862 (glufanide disodium, Cytran Inc. of Kirkland, Wash., USA), VEGFR2-selective monoclonal antibody DC101 (ImClone Systems, Inc.), angiozyme, a synthetic ribozyme from Ribozyme (Boulder, Colo.) and Chiron (Emeryville, Calif.), Sirna-027 (an siRNA-based VEGFR1 inhibitor, Sirna Therapeutics, San Francisco, Calif.) Caplostatin, soluble ectodomains of the VEGF receptors, Neovastat (AEterna Zentaris Inc; Quebec City, Calif.) and combinations thereof.

Other VEGF inhibitors useful in the practice of the present invention are disclosed in U.S. Pat. Nos. 6,534,524 and 6,235,764, both of which are incorporated in their entirety. Additional VEGF inhibitors are described in, for example in WO 99/24440 (published May 20, 1999), POT International Application PCT/IB99/00797 (filed May 3, 1999), in WO 95/21613 (published Aug. 17, 1995), WO 99/61422 (published Dec. 2, 1999), U.S. Pat. Publ. No. 20060094032 “iRNA agents targeting VEGF”, U.S. Pat. No. 6,534,524 (discloses AG13736), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), U.S. Pat. No. 6,653,308 (issued Nov. 25, 2003), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published April 8, 1999), and WO 98/024317(published Jan. 22, 1998), WO 01/02369 (published Jan. 11, 2001); U.S. Provisional Application No. 60/491,771 piled Jul. 31, 2003); U.S. Provisional Application No. 60/460,695 (filed Apr. 3, 2003); and WO 03/106462A1 (published Dec. 24, 2003). Additional examples of VEGF inhibitors are disclosed in International Patent Publications WO 99/62890 published Dec. 9, 1999, WO 01/95353 published Dec. 13, 2001 and WO 02/44158 published Jun. 6, 2002.

### Angiogenesis Screening Assays

Examples of angiogenesis screening assays that may be used to test the activity of agents for use in the invention include, but are not limited to, in vitro endothelial cell assays, rat aortic ring angiogenesis assays, cornea micropocket assays (corneal neovascularization assays), and chick embryo chorioallantoic membrane assays (Erwin, A. et al. (2001) Seminars in Oncology 28(6):570-576).

Some examples of in vitro endothelial cell assays include methods for monitoring endothelial cell proliferation, cell migration, or tube formation. Cell proliferation assays may use cell counting, BRdU incorporation, thymidine incorporation, or staining techniques (Montesano, R. (1992) Eur J Clin Invest 22:504-515; Montesano, R. (1986) Proc Natl. Acad. Sci. USA 83:7297-7301; Holmgren L. et al. (1995) Nature Med 1:149-153).

In the cell migration assays endothelial cells are plated on matrigel and migration monitored upon addition of a chemoattractant (Homgren, L. et al. (1995) Nature Med 1:149-153; Albini, A. et al. (1987) Cancer Res. 47:3239-3245; Hu, G. et al. (1994) Proc Natl Acad Sci USA 6:12096-12100; Alessandri, G. et al. (1983) Cancer Res. 43:1790-1797.)

The endothelial tube formation assays monitor vessel formation (Kohn, E C. et al. (1995) Proc Natl Acad Sci USA 92:1307-1311; Schnaper, H W. et al. (1995) J Cell Physiol 165:107-118).

Rat aortic ring assays have been used successfully for the screening of angiogenesis drugs (Zhu, W H. et al. (2000) Lab Invest 80:545-555; Kruger, E A. et al. (2000) Invasion Metastas 18:209-218; Kruger, E A. et al. (2000) Biochem Biophys Res Commun 268:183-191; Bauer, K S. et al. (1998) Biochem Pharmacol 55:1827-1834; Bauer, K S. et al. (2000) J Pharmacol Exp Ther 292:31-37; Berger, A C. et al. (2000) Microvasc Res 60:70-80.). Briefly, the assay is an ex vivo model of explant rat aortic ring cultures in a three dimensional matrix. One can visually observe either the presence or absence of microvessel outgrowths. The human saphenous angiogenesis assay, another ex-vivo assay, may also be used (Kruger, E A. et al. (2000) Biochem Biophys Res Commun 268:183-191).

Another common screening assay is the cornea micropocket assay (Gimbrone, M A. et al. (1974) J Natl Canc Inst. 52:413-427; Kenyon, B M. et al. (1996) Invest Opthalmol V is Sci 37:1625-1632; Kenyon, B M. et al. (1997) Exp Eye Res 64:971-978; Proia, A D. et al. (1993) Exp Eye Res 57:693-698). Briefly, neovascularization into an avascular space is monitored in vivo. This assay is commonly performed in rabbit, rat, or mouse.

The chick embryo chorioallantoic membrane assay has been used often to study tumor angiogenesis, angiogenic factors, and antiangiogenic compounds (Knighton, D. et al. (1977) Br J Cancer 35:347-356; Auerbach, R. et al. (1974) Dev Biol 41:391-394; Ausprunk, D H. et al. (1974) Dev Biol 38:237-248; Nguyen, M. et al. (1994) Microvasc Res 47:31-40). This assay uses fertilized eggs and monitors the formation of primitive blood vessels that form in the allantois, an extra-embryonic membrane.

The above is just a sampling of assays that may be used to assess the antiangiogenic activity of the anti-angiogenic agents or compounds to be used in methods of the invention.

There are a variety of diseases or disorders in which angiogenesis is believed to be important, referred to as angiogenic diseases including, but not limited to, obesity, inflammatory disorders such as immune and non-immune inflammation, chronic articular rheumatism and psoriasis, endometriosis, disorders associated with inappropriate or inopportune invasion of vessels such as diabetic retinopathy, macular degeneration, neovascular glaucoma, restenosis, capillary proliferation in atherosclerotic plaques and osteoporosis, and cancer associated disorders, such as solid tumors, solid tumor metastases, angiofibromas, retrolental fibroplasia, hemangiomas, Kaposi sarcoma and the like cancers which require neovascularization to support tumor growth.

As described herein, any of a variety of tissues, or organs comprised of organized tissues, can support angiogenesis in disease conditions including skin, muscle, gut, connective tissue, joints, bones and the like tissue in which blood vessels can invade upon angiogenic stimuli.

In one related embodiment, a tissue to be treated is an inflamed tissue and the angiogenesis to be inhibited is inflamed tissue angiogenesis where there is neovascularization of inflamed tissue. In this class the method contemplates inhibition of angiogenesis in arthritic tissues, such as in a patient with chronic articular rheumatism, in immune or non-immune inflamed tissues, in psoriatic tissue and the like.

In one embodiment, a tissue to be treated is a retinal tissue of a patient with a retinal disease such as diabetic retinopathy, macular degeneration or neovascular glaucoma and the angiogenesis to be inhibited is retinal tissue angiogenesis where there is neovascularization of retinal tissue.

In one embodiment, a tissue to be treated is a tumor tissue of a patient with a solid tumor, metastases, a skin cancer, a breast cancer, a medullary thyroid cancer, a hemangioma or angiofibroma and the like cancer, and the angiogenesis to be inhibited is tumor tissue angiogenesis where there is neovascularization of a tumor tissue. Tumors which may be treated by preventing or inhibiting angiogenesis with the combination therapy of the invention include, but are not limited to lung tumors, pancreas tumors, breast tumors, colon tumors, laryngeal tumors, ovarian tumors, thyroid tumors, melanoma, adenocarcinoma, sarcomas, thymoma, lymphoma, liver tumors, kidney tumors, non-Hodgkins lymphoma, Hodgkins lymphoma, leukemias, uterine tumors, prostate tumors, renal tumors, brain tumors, testicular tumors, bone tumors, muscle tumors, tumors of the placenta, gastric tumors and the like.

Disrupting tumor tissue angiogenesis is a particularly preferred embodiment because of the important role neovascularization plays in tumor growth. In the absence of neovascularization of tumor tissue, the tumor tissue does not obtain the required nutrients, slows in growth, ceases additional growth, regresses and ultimately becomes necrotic resulting in killing of the tumor.

Stated in other words, the present invention provides for a method of inhibiting tumor neovascularization by inhibiting tumor angiogenesis according to the present methods. Similarly, the invention provides a method of inhibiting tumor growth by practicing the angiogenesis-inhibiting methods.

The methods are also particularly effective against the formation of metastases because (1) their formation requires vascularization of a primary tumor so that the metastatic cancer cells can exit the primary tumor and (2) their establishment in a secondary site requires neovascularization to support growth of the metastases.

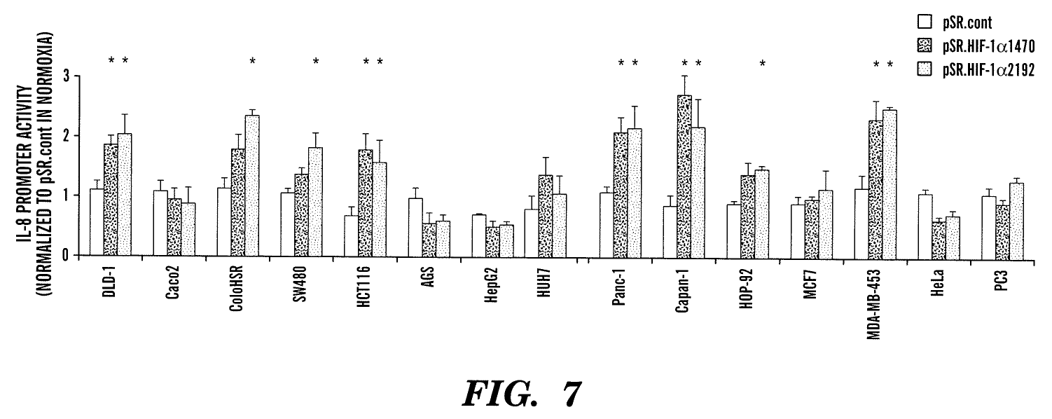

Preferably, when a combination therapy using both a HIF-1 inhibitor and an IL-8 used to inhibit angiogenesis in cancer, the cancer is either colon cancer, pancreatic cancer, lung cancer, or breast cancer (See Example 1, FIG. 7). In one embodiment, the cancer is either colon cancer, pancreatic cancer, or breast cancer.

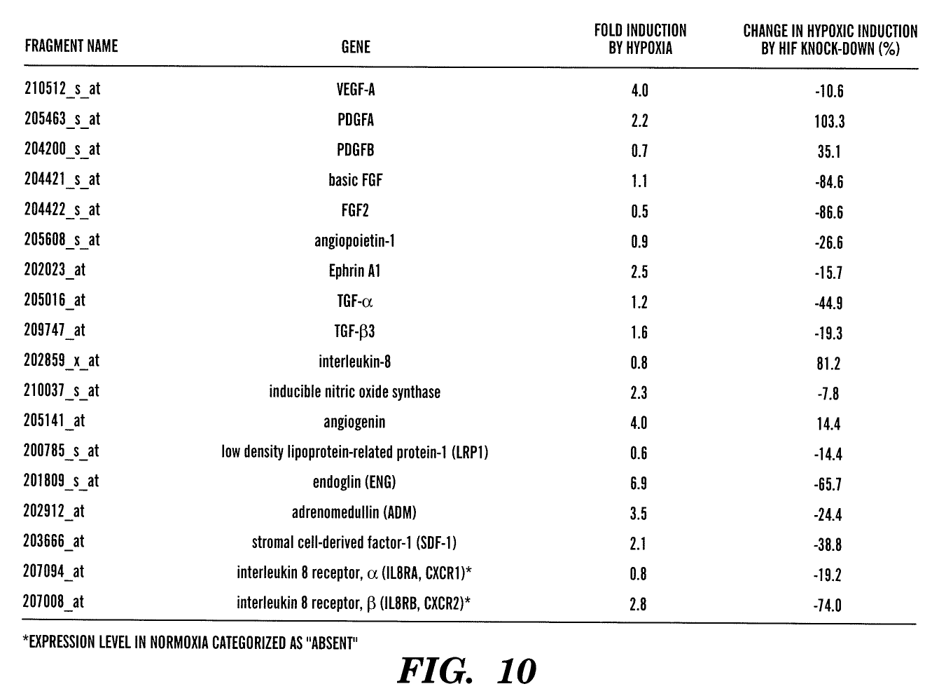

In one embodiment, when the combination therapy comprising a HIF-1 inhibitor is used to inhibit angiogenesis in colon cancer, the second anti-angiogenic agent is selected from the group consisting of an inhibitor of platelet derived growth factor (PDGF) (e.g. PDGF A or PDGF B), an inhibitor of IL-8, an inhibitor and an inhibitor of angiogenin (See Example 1, FIG. 10). Preferably, either an inhibitor of PDGF or IL-8 are used in combination with HIF-1 to treat colon cancer.

In one embodiment, when the combination therapy comprising a HIF-1 inhibitor is used to inhibit angiogenesis associated with colon cancer, a third anti-angiogenic agent is used. For example, a combination of an inhibitor of platelet derived growth factor (PDGF) (e.g. PDGF A or PDGF B), an inhibitor of IL-8, and an inhibitor of HIF-1.

In another embodiment, the invention contemplates the practice of the method in conjunction with other therapies such as conventional chemotherapy, radiation therapy or surgery directed against solid tumors and for control of establishment of metastases. The administration of angiogenesis-inhibiting amounts of combination therapy may be conducted before, during or after chemotherapy, radiation therapy or surgery.

For the combination therapy, the dose of the HIF-1 inhibitor may be administered prior to, concurrently, or after administration of a second compound or agent that is anti-angiogenic.

In the method of treatment, the administration of the combination therapy may be for either “prophylactic” or “therapeutic” purpose. When provided prophylactically, therapy is provided in advance of any symptom. The prophylactic administration of the combination therapy serves to prevent or inhibit an angiogenesis disease or disorder, i.e. cancer. Prophylactic administration of the combination therapy may be given to a patient with, for example, a family history of cancer. Alternatively, administration of the combination therapy may be given to a patient with rising cancer marker protein levels. Such markers include, for example, rising PSA, thymosin β-15, thymosin β-16, calcitonin, matrix metalloproteinase (MMP), and myeloma M-protein.

When provided therapeutically, the combination therapy is provided at (or after) the symptom or indication of angiogenesis. Thus, the combination therapy may be provided either prior to the anticipated angiogenesis at a site or after the angiogenesis has begun at a site.

Insofar as the present methods apply to inhibition of tumor neovascularization, the methods can also apply to inhibition of tumor tissue growth, to inhibition of tumor metastases formation, and to regression of established tumors.

Restenosis is a process of smooth muscle cell (SMC) migration and proliferation at the site of percutaneous transluminal coronary angioplasty which hampers the success of angioplasty. The migration and proliferation of SMC's during restenosis can be considered a process of angiogenesis which is disrupted by the present methods. Therefore, the invention also contemplates inhibition of restenosis by inhibiting angiogenesis according to the present methods in a patient following angioplasty procedures. For inhibition of restenosis, the combination therapy is typically administered after the angioplasty. The administration of the compounds of the invention may occur from about 2 to about 28 days post-angioplasty and more typically for about the first 14 days following the procedure.

The present method for inhibiting angiogenesis in a tissue, and therefore for also practicing the methods for treatment of angiogenesis-related diseases, comprises contacting a tissue in which angiogenesis is occurring, or is at risk for occurring, with a composition comprising a therapeutically effective amount of HIF-1 inhibitor together with a composition comprising a therapeutically effective amount of a second agent or compound that inhibits angiogenesis.

The HIF-1 inhibitor and second agent or compound that inhibits angiogenesis can be present in the same or different pharmaceutical composition.

The effective dosage range for the administration of the inhibitors depends upon the form of the inhibitor and its potency. It is an amount large enough to produce the desired effect in which angiogenesis and the disease symptoms mediated by angiogenesis are ameliorated. The dosage should not be so large as to cause adverse side effects, such as hyperviscosity syndromes pulmonary edema, congestive heart failure, and the like. Generally, the dosage will vary with the age, condition, sex and extent of the disease in the patient and can be determined by one of skill in the art. The dosage can also be adjusted by the individual physician in the event of any complication.

A therapeutically effective amount is an amount sufficient to produce a measurable inhibition of angiogenesis or tumor growth in the tissue being treated, i.e., an angiogenesis-inhibiting amount. Inhibition of angiogenesis can be measured in situ by immunohistochemistry, or by other methods known to one skilled in the art.

One skilled in the art can readily assess the potency of the candidate combined therapy of this invention.

In general, it is desirable to provide the recipient with a dosage of each inhibitor of at least about 10 μg/kg, preferably at least about 10 mg/kg or higher. A range of from about 1 μg/kg to about 100 mg/kg is preferred although a lower or higher dose may be administered. The dose is administered at least once and may be provided as a bolus, a continuous administration or sustained release. Multiple administration over a period of weeks or months may be preferable. It may also be preferable to administer the dose at least once/week and even more frequent administrations (e.g. daily). Subsequent doses may be administered as indicated.

The route of administration may be intravenous (I.V.), intramuscular (I.M.), subcutaneous (S.C.), intradermal (I.D.), intraperitoneal (I.P.), intrathecal (I.T.), intrapleural, intrauterine, rectal, vaginal, topical, intratumor and the like. The inhibitors of the invention can be administered parenterally by injection or by gradual infusion over time and can be delivered by peristaltic means.

Administration may be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. In addition, detergents may be used to facilitate permeation. Transmucosal administration may be through nasal sprays, for example, or using suppositories. For oral administration, the compounds of the invention are formulated into conventional oral administration forms such as capsules, tablets and tonics.

For topical administration, the dose is formulated into ointments, salves, gels, or creams, as is generally known in the art.

The combined therapy compositions are administered in a manner compatible with the dosage formulation, and in a therapeutically effective amount. The quantity to be administered and timing depends on the subject to be treated, capacity of the subject's system to use the active ingredient, and degree of therapeutic effect desired. Precise amounts of active ingredient required to be administered depend on the judgment of the practitioner and are peculiar to each individual.

### Therapeutic Compositions

Any formulation or drug delivery system containing the active ingredients, which is suitable for the intended use, as are generally known to those of skill in the art, can be used. Suitable pharmaceutically acceptable carriers for oral, rectal, topical or parenteral (including inhaled, subcutaneous, intraperitoneal, intramuscular and intravenous) administration are known to those of skill in the art. The carrier must be pharmaceutically acceptable in the sense of being compatible with the other ingredients of the formulation and not deleterious to the recipient thereof.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects.

Formulations suitable for parenteral administration conveniently include sterile aqueous preparation of the active compound which is preferably isotonic with the blood of the recipient. Thus, such formulations may conveniently contain distilled water, 5% dextrose in distilled water or saline. Useful formulations also include concentrated solutions or solids containing the compound which upon dilution with an appropriate solvent give a solution suitable for parental administration above.

For enteral administration, a compound can be incorporated into an inert carrier in discrete units such as capsules, cachets, tablets or lozenges, each containing a predetermined amount of the active compound; as a powder or granules; or a suspension or solution in an aqueous liquid or non-aqueous liquid, e.g., a syrup, an elixir, an emulsion or a draught. Suitable carriers may be starches or sugars and include lubricants, flavorings, binders, and other materials of the same nature.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

Formulations for rectal administration may be presented as a suppository with a conventional carrier, e.g., cocoa butter or Witepsol S55 (trademark of Dynamite Nobel Chemical, Germany), for a suppository base.

Formulations for oral administration may be presented with an enhancer. Orally-acceptable absorption enhancers include surfactants such as sodium lauryl sulfate, palmitoyl carnitine, Laureth-9, phosphatidylcholine, cyclodextrin and derivatives thereof; bile salts such as sodium deoxycholate, sodium taurocholate, sodium glycochlate, and sodium fusidate; chelating agents including EDTA, citric acid and salicylates; and fatty acids (e.g., oleic acid, lauric acid, acylcarnitines, mono- and diglycerides). Other oral absorption enhancers include benzalkonium chloride, benzethonium chloride, CHAPS (3-(3-cholamidopropyl)-dimethylammonio-1-propanesulfonate), Big-CHAPS (N,N-bis(3-D-gluconamidopropyl)-cholamide), chlorobutanol, octoxynol-9, benzyl alcohol, phenols, cresols, and alkyl alcohols. An especially preferred oral absorption enhancer for the present invention is sodium lauryl sulfate.

Alternatively, the compound may be administered in liposomes or microspheres (or microparticles). Methods for preparing liposomes and microspheres for administration to a patient are well known to those of skill in the art. U.S. Pat. No. 4,789,734, the contents of which are hereby incorporated by reference, describes methods for encapsulating biological materials in liposomes. A review of known methods is provided by G. Gregoriadis, Chapter 14, “Liposomes,” Drug Carriers in Biology and Medicine, pp. 287-341 (Academic Press, 1979).

Microspheres formed of polymers or proteins are well known to those skilled in the art, and can be tailored for passage through the gastrointestinal tract directly into the blood stream. Alternatively, the compound can be incorporated and the microspheres, or composite of microspheres, implanted for slow release over a period of time ranging from days to months. See, for example, U.S. Pat. Nos. 4,906,474, 4,925,673, and 3,625,214, and Jein, TIPS 19:155-157 (1998), the contents of which are hereby incorporated by reference.

In one embodiment, the inhibitor can be formulated into a liposome or microparticle which is suitably sized to lodge in capillary beds following intravenous administration. When the liposome or microparticle is lodged in the capillary beds surrounding ischemic tissue, the agents can be administered locally to the site at which they can be most effective. Suitable liposomes for targeting ischemic tissue are generally less than about 200 nanometers and are also typically unilamellar vesicles, as disclosed, for example, in U.S. Pat. No. 5,593,688 to Baldeschweiler, entitled “Liposomal targeting of ischemic tissue,” the contents of which are hereby incorporated by reference.

Preferred microparticles are those prepared from biodegradable polymers, such as polyglycolide, polylactide and copolymers thereof. Those of skill in the art can readily determine an appropriate carrier system depending on various factors, including the desired rate of drug release and the desired dosage.

In one embodiment, the formulations are administered via catheter directly to the inside of blood vessels. The administration can occur, for example, through holes in the catheter. In those embodiments wherein the active compounds have a relatively long half life (on the order of 1 day to a week or more), the formulations can be included in biodegradable polymeric hydrogels, such as those disclosed in U.S. Pat. No. 5,410,016 to Hubbell et al. These polymeric hydrogels can be delivered to the inside of a tissue lumen and the active compounds released over time as the polymer degrades. If desirable, the polymeric hydrogels can have microparticles or liposomes which include the active compound dispersed therein, providing another mechanism for the controlled release of the active compounds.

The formulations may conveniently be presented in unit dosage form and may be prepared by any of the methods well known in the art of pharmacy. All methods include the step of bringing the active compound into association with a carrier which constitutes one or more accessory ingredients. In general, the formulations are prepared by uniformly and intimately bringing the active compound into association with a liquid carrier or a finely divided solid carrier and then, if necessary, shaping the product into desired unit dosage form.

The formulations may further include one or more optional accessory ingredient(s) utilized in the art of pharmaceutical formulations, e.g., diluents, buffers, flavoring agents, binders, surface active agents, thickeners, lubricants, suspending agents, preservatives (including antioxidants) and the like.

The inhibitors may be presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns, more preferably between 2 and 5 microns.

A formulation for the administration of protein via the nasal route is described in U.S. Pat. No. 5,759,565, and can be modified for the inhibitors described herein. This nasal formulation is designed to be stored in a multi-dose container, is stable for an extended period of time, and resists bacterial contamination. The preservative in the formulation, benzalkonium chloride, enhances the absorption of the protein.

Generally for nasal administration a mildly acid pH will be preferred. Preferably the compositions of the invention have a pH of from about 3 to 5, more preferably from about 3.5 to about 3.9 and most preferably 3.7. Adjustment of the pH is achieved by addition of an appropriate acid, such as hydrochloric acid.

The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions, in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The inhibitors of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Exemplary of such additional liquid phases are glycerin, vegetable oils such as cottonseed oil, and water-oil emulsions.

### EXAMPLES

### Example I

### Induction of Interleukin-8 Preserves the Angiogenic Response in HIF-1 Deficient Colon Cancer Cells

### Methods

Cell lines. DLD-1 and Caco2 cells (ATCC) were stably transfected with HIF-1α siRNA constructs (pSuper.retro, OligoEngine), pSR.HIF-1α1470 or pSR.HIF-1α2192¹⁰. Three independent DLD-1 clones stably expressing pSR.HIF-1α1470 and two independent clones expressing pSR.HIF-1α2192 exhibited similar responses to hypoxia with respect to induction of NF-κB and IL-8. In a pilot xenograft study, growth, microvascular density, VEGF and IL-8 levels were similar between a pSR.HIF-1α1470 clone and pSR.HIF-1α2192 clone. Hypoxic conditions (1% O₂) were achieved with a sealed hypoxia chamber (Billups-Rothenberg) in serum free UltraCulture medium (Cambrex)¹⁰. Transient transfections were performed using Lipofectamine 2000 (Invitrogen).

Plasmid constructs. The IL-8 reporter 26, NF-κB reporter, and phr-GFP-K-ras^{V12} plasmids have been described²⁷. Site directed mutagenesis was performed to obtain the phr-GFP-Kras^{D13} construct pSuper.K-rasD13 (pSR.K-ras^{D13}) was constructed by subcloning the sequence 5-GGAGCTGGTGACGTAGGCA (SEQ ID NO: 1). For control siRNA, pSR.cont, the sequence 5′-GCGCGCTTTGTAGGATTCG (SEQ ID NO: 2) was utilized²⁸.

Transfections and Reporter Assays. 0.1-0.2 μg reporter constructs were co-transfected with 2 ng of pRL-CMV (Promega) and luciferase activity was measured with the Dual Luciferase Reporter Assay System (Promega). pRL-null, a promoter-less Renilla construct, was used when cells were co-transfected with a K-ras expression vector²⁹. The relative luciferase activity was calculated as Firefly/Renilla luciferase activity. The level of ‘hypoxic induction’ was the ratio between the relative luciferase activity in hypoxia to that in normoxia.

Xenograft tumor model. 2×10⁶ cells were injected subcutaneously into the flanks of 6-8-week CD1 female nude mice (6 mice/arm). Tumors were measured with calipers and volume was calculated as [length×width²]×0.5. Neutralizing antibody to IL-8 (MAB208, clone 6217.111; R&D Systems) and/or VEGF (MAB293, R&D Systems) was administered i.p when tumors reached 5 mm. 100 μg of MAB208 and/or 25 μg of MAB293 was injected on days 7, 9, 11, 14, 16, 18, 21, and 23, before mice were sacrificed at day 25. To assess hypoxic regions, mice were injected with 60 mg/kg pimonidazol hydrochloride (Hypoxyprobe-1, Chemicon) i.p., 1.5 hr before sacrifice. To visualize functional tumor microvessels, 100 μg FITC-labeled tomato lectin (Vector Laboratories) was injected i.v., and mice were heart-perfused with 4% paraformaldehyde. This protocol was approved by the Animal Care and Use Committee of the Massachusetts General Hospital.

Immunohistochemistry. 5 μm sections from fresh frozen tumors were treated with acetone and endogenous peroxidase was blocked with 3% H₂O₂. The sections were incubated with a CD31 antibody, MEC13.3 (1:50; Phanningen) overnight at 4° C. Blood vessels were counted in 5-10 random viable fields (200×). To detect tumor hypoxia, formalin fixed sections were treated with 0.01% pronase and incubated with Hypoxpyrobe-1 antibody Mab1 (1:50; Chemicon). For other immunohistochemical studies, xenograft tissues were fixed in 10% neutral buffered formalin. TUNEL staining was performed with the ApoAlert DNA fragmentation detection kit (Clontech). Ki-67 staining was performed with the MIB-1 antibody (1:100; DAKO) and staining for phospho-p65 (Ser 563) (1:50; Cell Signaling) was also performed.

Real-time PCR assay. RNA was extracted using the RNeasy kit (Qiagen) and quantitative reverse-transcription PCR was performed using the SuperScript III platinum Two-Step qRT-PCR Kit (Invitrogen). Primer sequences for VEGF, IL-8, and 18S RNA are available upon request. A fluorogenic SYBR Green and MJ research detection system were used for real time quantification.

Immunoblotting. Immunoblot analysis for HIF-1α (clone 54, 1:250; Transduction Laboratories), HIF-2α (1:250, Novus), Glut-1 (GT-11A, 1:1000; Alpha Diagnostic International), VEGF (Ab-2, 1:40; Calbiochem), phospho (Ser 563)- and total-NF-κB p65 (1:1000; both Cell Signaling), K-ras (F234, 1:200; Santa Cruz) and β-actin (AC15, 1 μg/mL; Sigma) were performed after SDS-PAGE and electrophoretic transfer to PVDF membranes¹⁰.

ELISA. VEGF and IL-8 protein levels of conditioned medium and tissue lysates were assayed utilizing specific ELISA kits (Quantikine, R&D Systems).

Microarray analysis. Sample preparation and processing procedures were performed as described in the Affymetrix GeneChip Expression Analysis Manual (Santa Clara). The labeled cRNA samples were hybridized to the complete Affymetrix human U133 GeneChip set (HG-U133A).

Hydrogen peroxide studies. Measurement of H₂O₂ was performed using the Amplex Red Hydrogen peroxide Assay Kit and the CM-H2DCFDA reagent (both Molecular Probe). Cells were exposed to hypoxia for 10 h, and then culture medium was switched to Krebs-Ringer phosphate buffer³⁰ containing 100 μM Amplex Red reagent and 0.2 U/mL HRP. After additional incubation in hypoxia for 1 h, fluorescence was measured in 96-well plates by Spectra MAX GEMINI XS microplate fluorometer (Molecure Devices). Cells were also incubated with 10 μM CM-H2DCFDA for 30 minutes in RPMI without phenol red. Fluorescence was measured in 96 well plates and values were normalized to cell number. 20 or 40 μM t-butyl hydroperoxide (t-BH, Sigma) was added to the culture media of DLD-1 cells every 30 minutes for 6 hours, and IL-8 mRNA levels were measured by qRT-PCR.

Statistical analysis. Statistical analyses were performed with a two-tailed, unpaired Student's t-test.

Specificity of the pSR.HIF-1α1470 and pSR-HIF-1α2192 constructs. Specificity was demonstrated by co-expression of HIF-1α expression vectors with synonymous codon mutations that are not affected by the siRNA target sequences. (Primers: SDM-HIF1470 forward 5′-AAA TTA GAA CCA AAT CCA GAA AGC CTG GAA CTT TCT TTT ACC ATG C (SEQ ID NO: 3), SDM-HIF1470 reverse 5′-GCA TGG TAA AAG AAA GTT CCA GGC TTT CTG GAT TTG GTT CTA ATT T (SEQ ID NO: 4). SDM-HIF2192 forward 5′-GAA AAA TGG AAC ATG ATG GCA GCC TTT TTC AAG CAG TAG GAA TTG G (SEQ ID NO: 5). SDM-HIF2192 reverse 5′-CCA ATT CCT ACT GCT TGA AAA AGG CTG CCA TCA TGT TCC ATT TTT C (SEQ ID NO: 6). The underlined sequence is targeted by the siRNA construct, and the bold nucleotides indicate the point mutations introduced.)

### Results

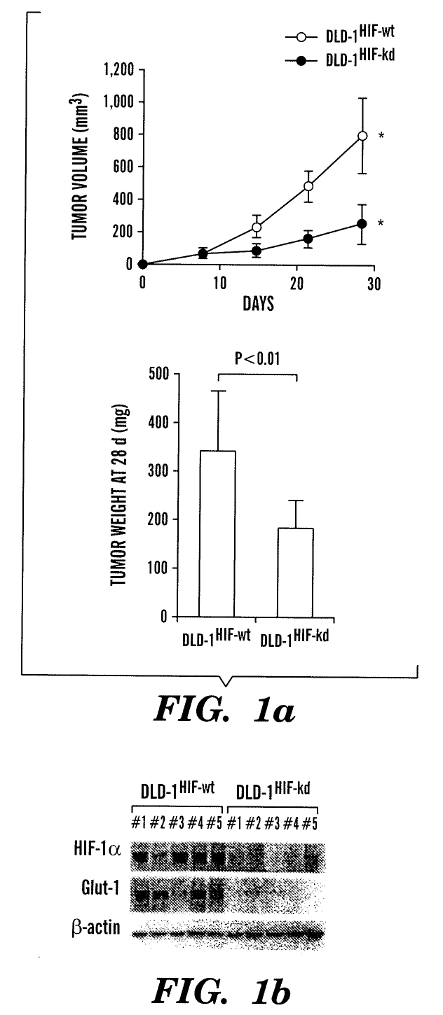

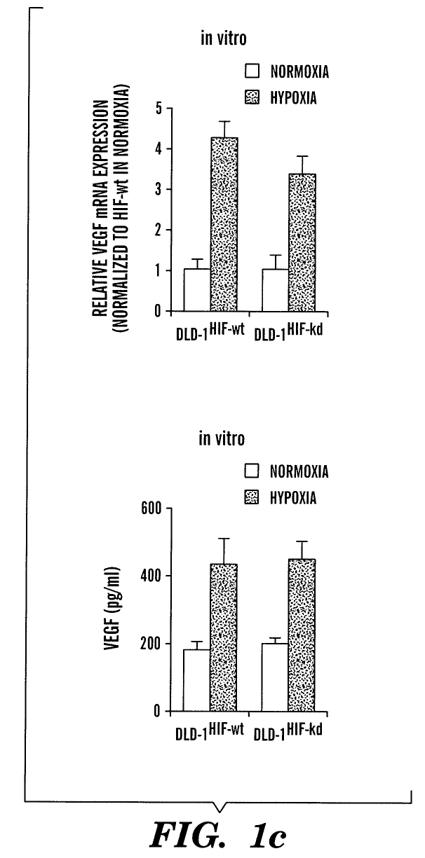

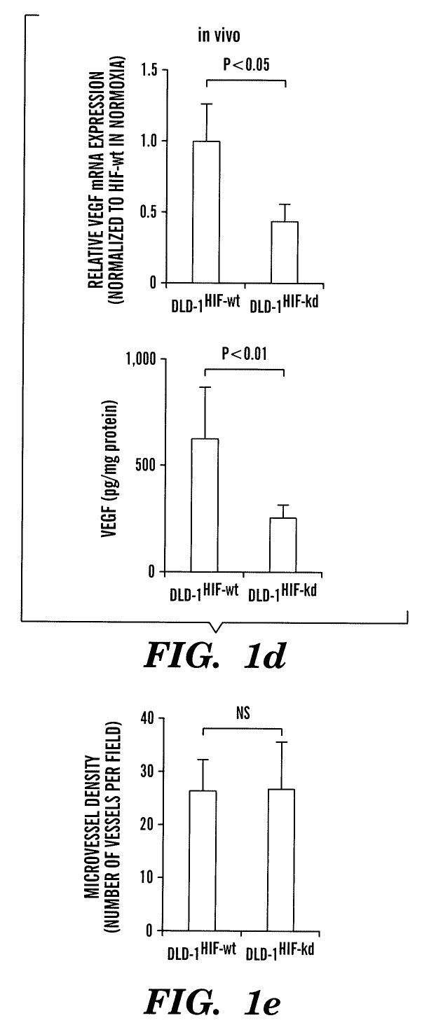

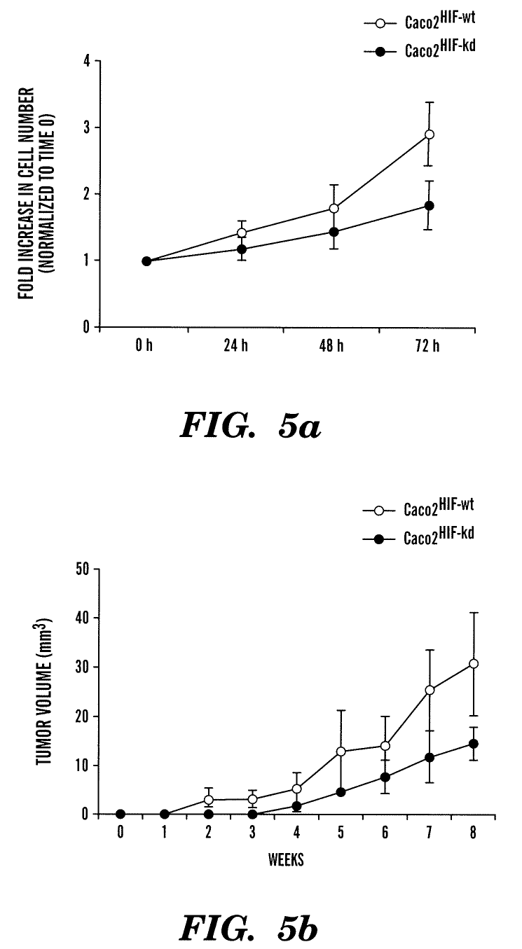

DLD-1 cells, either with or without HIF-1α stably knocked-down by siRNA¹⁰ (DLD-1^{HIF-kd} or DLD-1^{HIF-wt}, respectively), were injected subcutaneously into CD1 nude mice. Four weeks after inoculation, both tumor volumes and weights were significantly lower in DLD-1^{HIF-kd} tumors (FIG. 1A), indicating an important role for HIF-1 in tumor growth in vivo. We confirmed this finding in an independent colon cancer cell line, Caco2 (FIG. 5). Large necrotic areas were much more prevalent in DLD-1^{HIF-wt} xenografts (data not shown). Furthermore, a prominent inflammatory infiltrate composed predominantly of neutrophils was observed only in DLD-1^{HIF-kd} xenografts (data not shown). Although there were larger areas of necrosis in DLD-1^{HIF-wt} xenografts, the cross-sectional surface area of non-necrotic viable tumor was still significantly greater when compared to DLD-1^{HIF-kd} xenografts (0.33 cm² vs. 0.16 cm², respectively, P=0.025). Thus, the difference in size of the tumors cannot be entirely attributed to the larger area of necrosis in the DLD-1^{HIF-wt} tumors. A persistent silencing effect of the siRNA/HIF-1α construct was confirmed in vivo (FIG. 1B).

There was a significant decrease in the Ki-67 labeling index in DLD-1^{HIF-kd} xenografts (41.3±3.2% in DLD-1^{HIF-wt} tumors vs. 27.4±2.6% in DLD-1^{HIF-kd} tumors; P<0.01), suggesting that HIF-1α regulates cellular proliferation in vivo. The apoptotic index was calculated by counting TUNEL positive cells in non-necrotic areas. A small but statistically significant difference in the apoptotic index was observed between the two groups (3.2±0.53% in DLD-1^{HIF-wt} tumors vs. 1.9±0.42% in DLD-1^{HIF-kd} tumors; P<0.05), but this difference is unlikely to counterbalance the dramatic difference in proliferation rates.

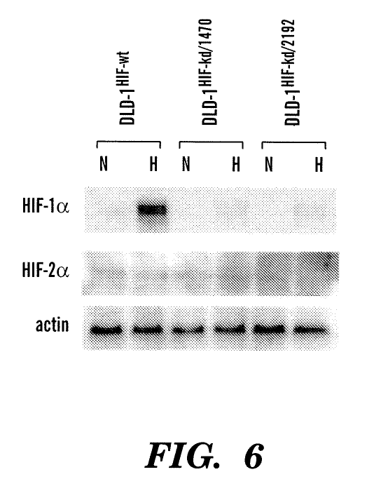

When DLD-1^{HIF-kd} cells were incubated in hypoxic conditions (1% O₂) in vitro, there was only a 25% reduction (P=0.11) in the induced levels of VEGF mRNA and protein (FIG. 1C). In the DLD-1^{HIF-kd} xenografts, VEGF mRNA and protein levels were also induced (FIG. 1D), though not to the same extent observed in vitro. Compared to the DLD-1^{HIF-wt} xenografts, VEGF mRNA levels were 51% lower (P=0.028) and protein levels were 52% lower (P=0.0024) in DLD-1^{HIF-kd} xenografts. This persistent expression of VEGF was not mediated by HIF-2α, as HIF-2α mRNA and protein levels were barely detectable in normoxic conditions and the gene was not induced by hypoxia (FIG. 6).

To specifically address whether hypoxia regulates VEGF in the absence of HIF-1 in vivo, hypoxic areas within the tumor mass were identified utilizing Hypoxyprobe-1 (pimonidazole hydroxychloride). There were large hypoxic regions surrounding the necrotic areas in the center of the DLD-1^{HIF-wt} tumors (data not shown). In contrast, DLD-1^{HIF-kd} tumors revealed only restricted regions of intratumoral hypoxia. Double immunofluorescence demonstrated that VEGF was preferentially expressed in the hypoxic areas of both DLD-1^{HIF-kd} and DLD-1^{HIF-wt} xenografts (data not shown).

It is possible that the difference in growth between the xenografts was due to impaired angiogenesis, potentially attributable to lower levels of VEGF in DLD-1^{HIF-kd} tumors. However, immunostaining for the endothelial cell marker CD31 revealed abundant microvascular networks in all tumors (FIG. 1E). No quantitative difference in microvessel density was observed (26.1±6.3/field in DLD-1^{HIF-wt} and 28.7±8.6/field in DLD-1^{HIF-kd} xenografts), suggesting that high levels of HIF-1 may not be required to stimulate angiogenesis or maintain vessel integrity in DLD-1 tumors.

Although up-regulation of VEGF was preserved in DLD-1^{HIF-kd} xenografts, the absolute levels of VEGF were reduced. We therefore determined whether other angiogenic factors may be induced in a compensatory manner to maintain tumor vascularity in the absence of HIF-1. cDNA microarray analysis identified genes that were up-regulated at least 2-fold by hypoxia but whose expression was attenuated less than 30% when HIF-1 was silenced. VEGF was up-regulated 4-fold in DLD-1^{HIF-wt} cells by hypoxia, and this induction was decreased only 10.6% by HIF-1 silencing (FIG. 10). In addition, expression of the pro-angiogenic cytokine IL8 was increased two-fold in DLD-1^{HIF-kd} cells cultured in hypoxic conditions compared to DLD-1^{HIF-wt} cells.

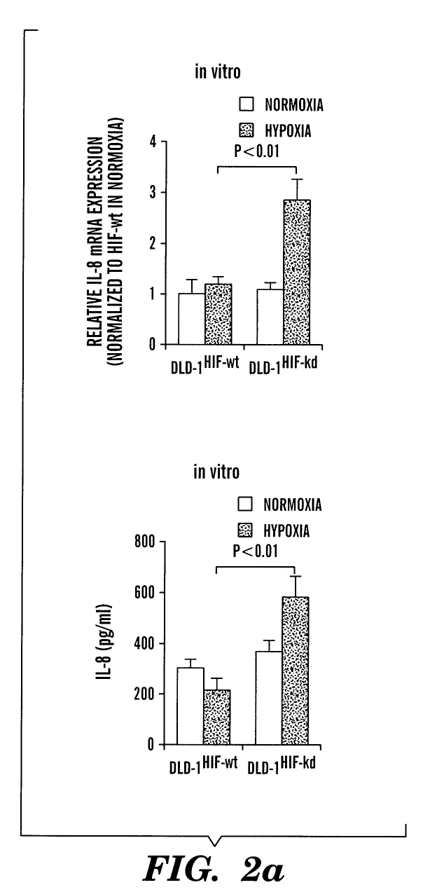

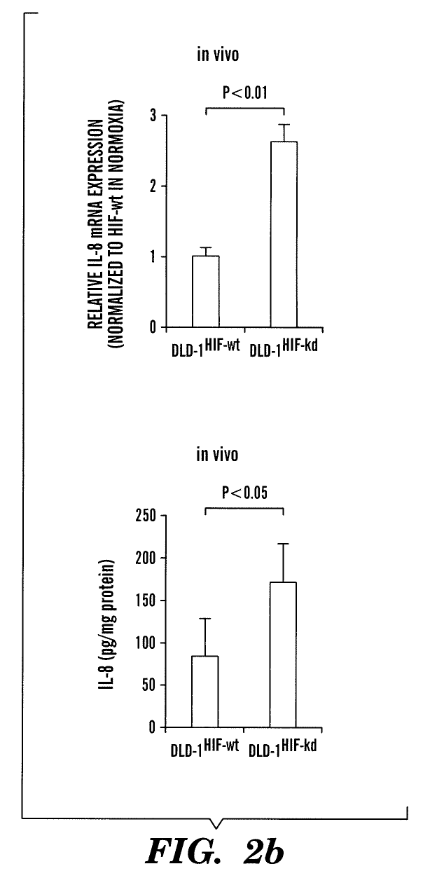

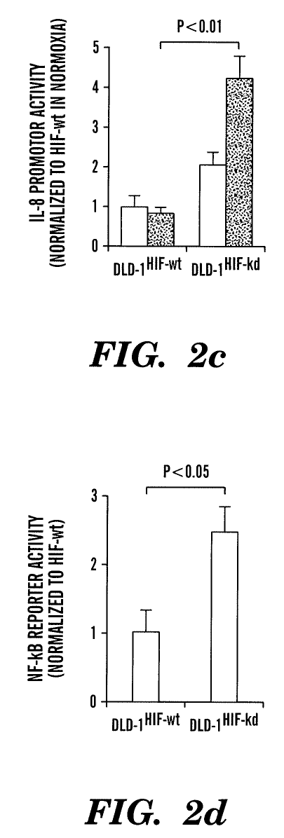

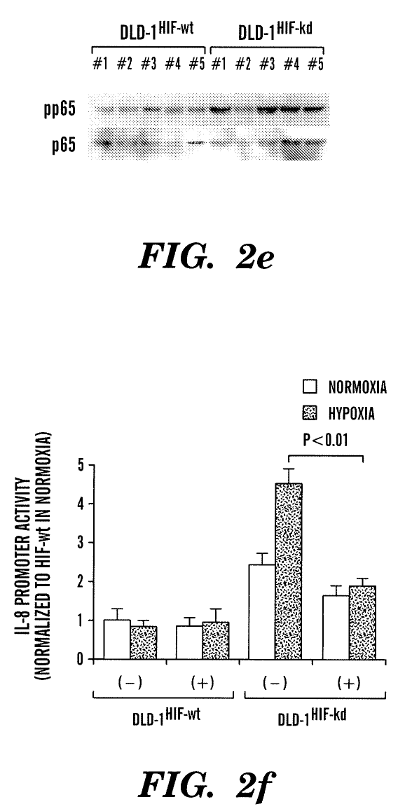

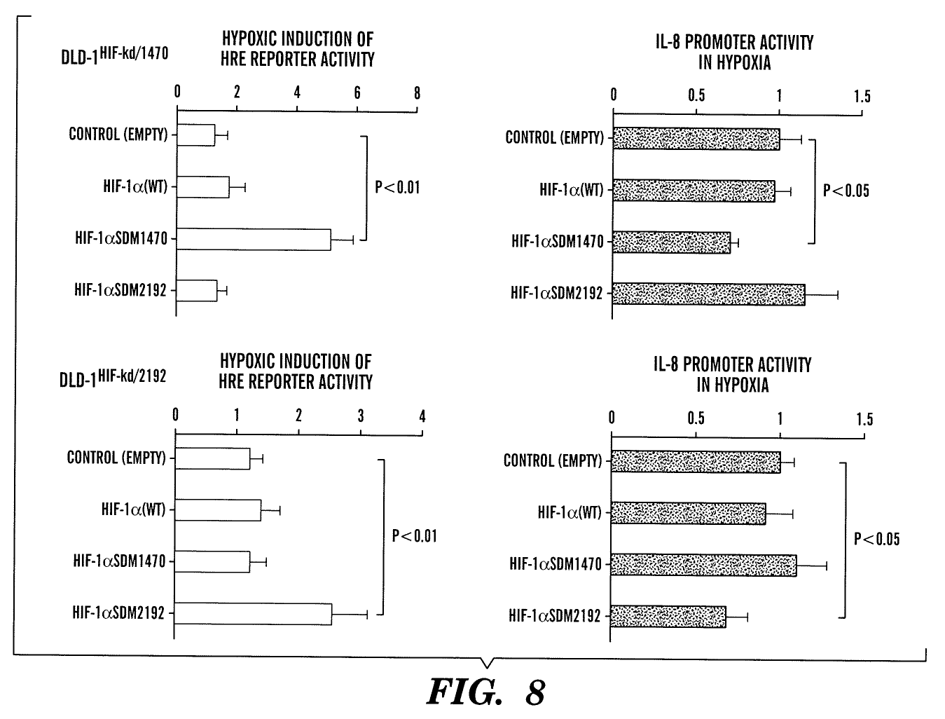

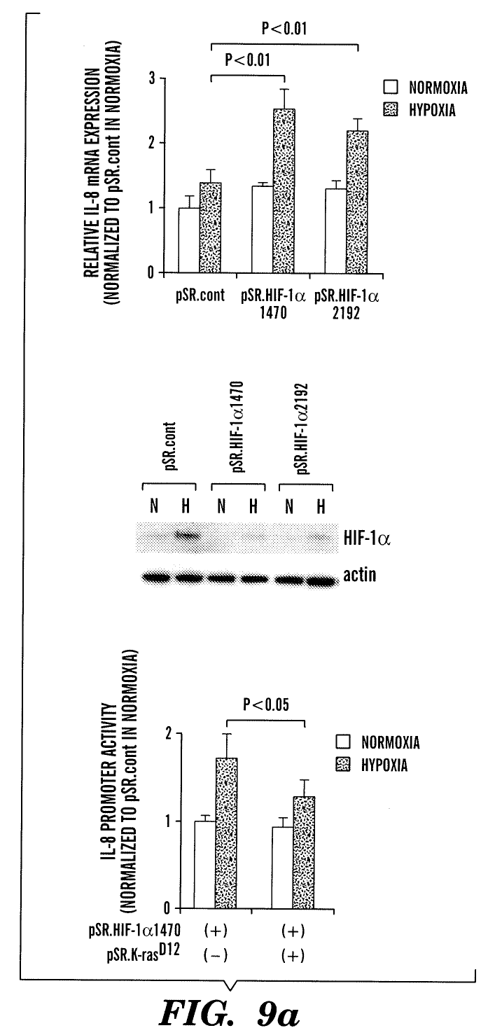

Hypoxia up-regulated IL8 mRNA more than 2.5-fold in DLD-1^{HIF-kd} cells, but there was no induction in DLD-1^{HIF-wt} cells (FIG. 2A). Consistent with this result, the IL-8 level in the supernatant of DLD-1^{HIF-kd} cells was increased almost 3-fold compared to DLD-1^{HIF-wt} cells. Similar results were obtained with independent DLD-1^{HIF-kd} clones previously established¹⁰ (data not shown). Extracts from DLD-1^{HIF-kd} xenografts also revealed significantly higher IL-8 mRNA and protein levels when compared to DLD-1^{HIF-wt} tumors (FIG. 2B). IL8 promoter reporter constructs exhibited higher basal activity in DLD-1^{HIF-kd} cells (FIG. 2C), and there was further induction of promoter activity in hypoxia that was not observed in the DLD-1^{HIF-wt} cells. There was also a 2.1-fold induction of the IL8 promoter when HIF-1α was transiently knocked-down in parental DLD-1 cells, indicating this phenomenon was not an artefact of the stable transfection process. In addition, expression of a constitutively active HIF-1α/P564A in DLD-1 cells failed to induce the IL8 promoter (1.01+/−0.14 fold increase), indicating that HIF-1 does not directly regulate IL8 gene expression. This hypoxic effect was not unique to DLD-1 cells. Knock-down of HIF-1α in additional colon cancer cells (ColoHSR, SW 480, and HCT116), pancreatic cancer cells (Panc-1, CAPAN-1), breast cancer cells (MDA-MB-453), and lung cancer cells (HOP-92) revealed a similar induction of IL-8 in hypoxia (FIG. 7). Finally, specificity of these siRNA constructs was confirmed by expression of HIF-1α synonymous codon mutants (FIG. 8). The absence of HIF-1 can therefore stimulate IL-8 on a transcriptional level, and this is further enhanced in hypoxia.

NF-κB is a major regulator of IL-8. NF-κB reporter activity was increased 151% (P<0.01) in HIF-1α knock-down cells (FIG. 2D). Western blotting (FIG. 2E) and immunohistochemistry (data not shown) of tissue xenografts revealed that phosphorylation of the p65 subunit was greater in DLD-1^{HIF-kd} xenografts, suggesting that HIF-1 inhibition does up-regulate the NF-κB pathway in vivo. Densitometry of western blots quantified a 2.0±0.4 fold increase in the ratio of phospho-p65/p65 (P<0.01). The hypoxic induction of the IL8 promoter in DLD-1^{HIF-kd} cells was significantly down-regulated by BAY 11-7082, a specific NF-κB inhibitor¹¹ (FIG. 2F). Thus, activation of the NF-κB pathway is important for the induction of IL-8 in the absence of HIF-1.

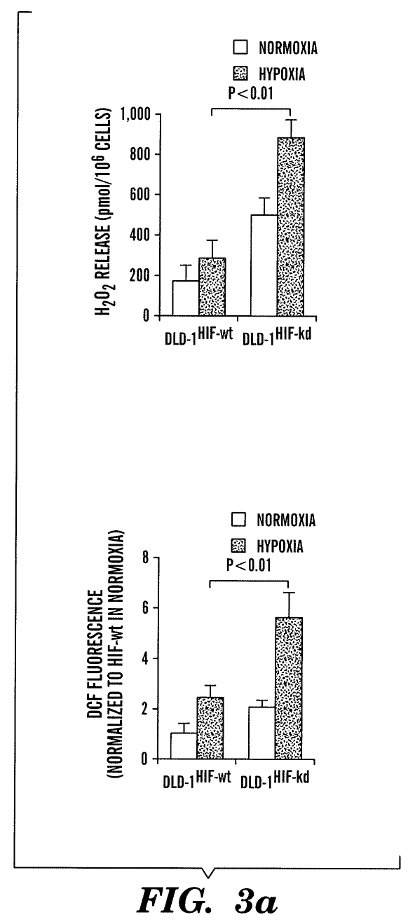

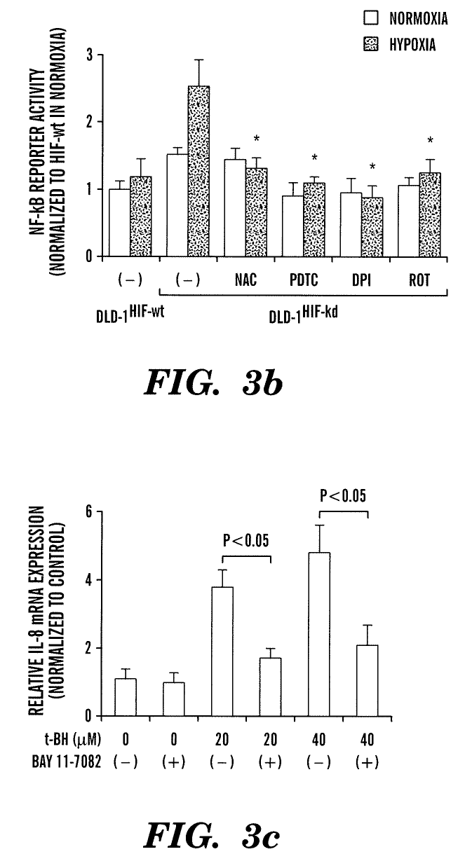

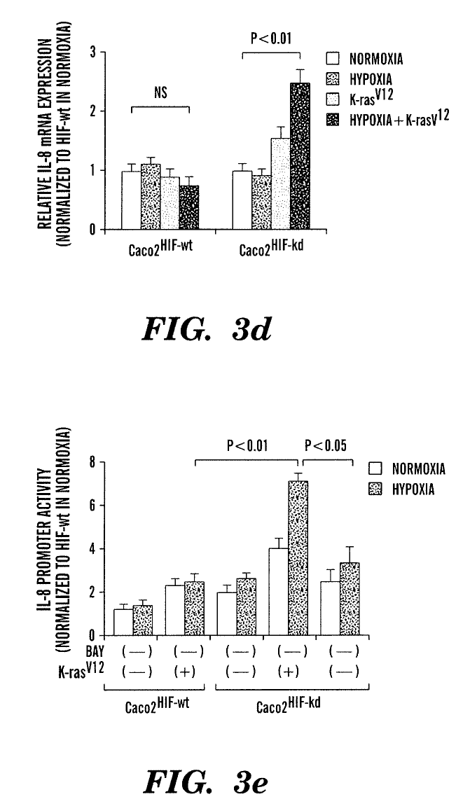

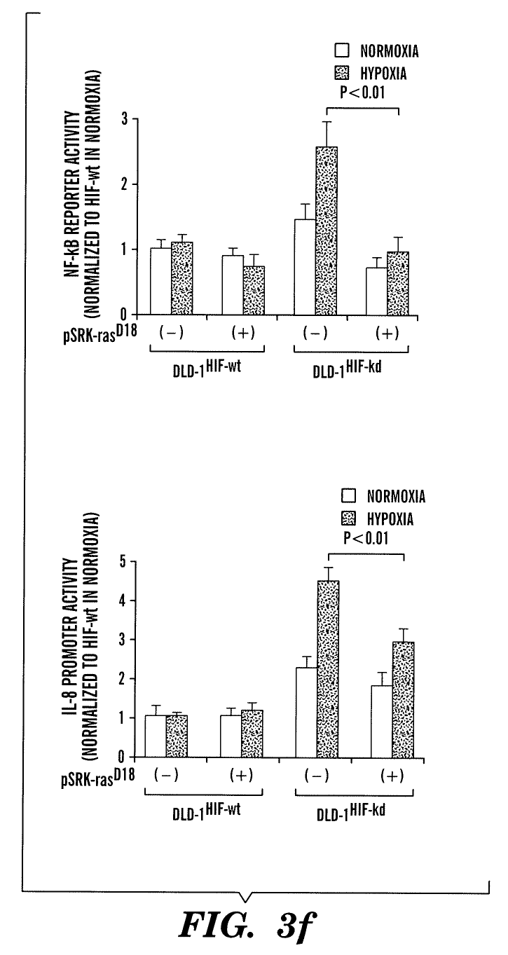

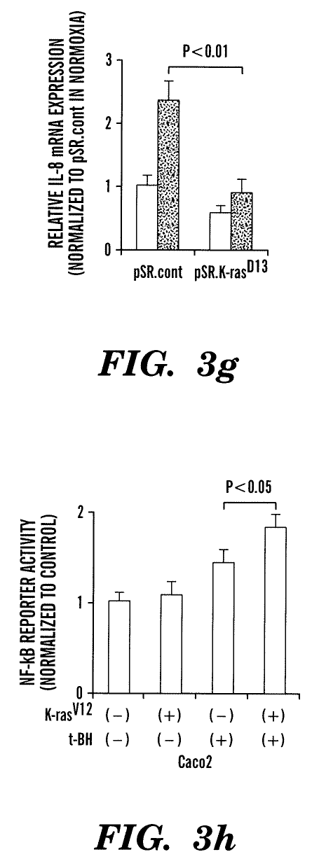

We then studied if HIF-1 inhibition may enhance the production of hydrogen peroxide, a reactive oxygen species (ROS) that can activate NF-κB^{12,13}. Hypoxic conditions can lead to the increased production of ROS^{14,15}, and scavenging of ROS is often achieved by increased production of pyruvate¹⁶ that occurs when cells shift from oxidative to glycolytic metabolism. This shift depends upon HIF-1α¹⁷. DLD-1^{HIF-kd} cells released more hydrogen peroxide in vitro, and hypoxia further enhanced its production (FIG. 3A). Four distinct chemical inhibitors of ROS production (N-acetyl-L-cysteine (NAC), pyrrolidinedithiocarbamate (PDTC), rotenone (Rot), and diphenylene iodonium (DPI)) each strongly blocked the induction of NF-κB promoter activity by hypoxia in DLD-1^{HIF-kd} cells (FIG. 3B). Finally, exogenous administration of the long-acting H₂O₂ analogue, t-butyl hydroperoxide (t-BH), stimulated the production of IL-8 in parental DLD-1 cells. This induction was inhibited by BAY 11-7082 (FIG. 3C), again demonstrating that NF-κB mediates this effect of ROS.

In contrast to DLD-1^{HIF-kd} cells, hypoxic induction of IL-8 mRNA (FIG. 3D) and protein (data not shown) was not observed in Caco2^{HIF-kd} colon cancer cells₁₀. Since DLD-1 cells harbor a mutant KRAS oncogene (KRAS^{D13}) whereas Caco2 cells are wild-type (KRAS^{WT}), we speculated that oncogenic KRAS may play a role in the hypoxic induction of IL-8¹⁸. When oncogenic KRAS^{V12} was expressed in Caco2^{HIF-kd} cells, hypoxia up-regulated IL8 mRNA 2.5-fold, whereas the effect was not observed in Caco2^{HIF-wt} cells or in Caco2^{HIF-kd} cells exposed to hypoxia only (FIG. 3D). KRAS^{V12} only modestly induced IL8 mRNA in Caco2^{HIF-kd} cells in normoxic conditions. Expression of KRAS^{V12} in Caco2^{HIF-wt} cells also up-regulated the IL8 promoter, but this activation was more pronounced in Caco2^{HIF-kd} cells in hypoxia (FIG. 3E). BAY 11-7082 blocked the induction of the IL8 promoter by hypoxia and KRAS^{V12} (FIG. 3E).

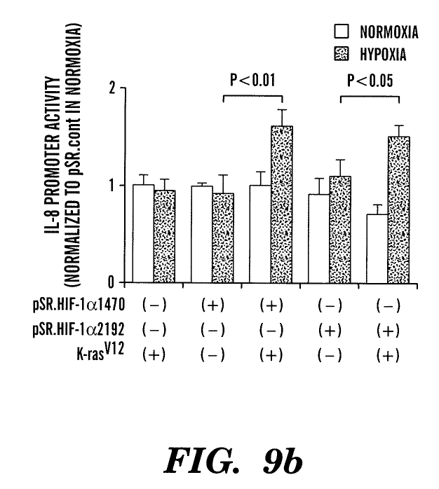

Exogenous expression of oncogenic KRAS may act supra-physiologically. Endogenous KRAS^{D13} in DLD-1 cells was therefore silenced by siRNA and this resulted in a 50% reduction of KRAS protein levels, consistent with a silencing effect of the one mutant allele¹⁹. Knock-down of KRAS^{D13} attenuated the hypoxic induction of NF-κB and IL-8 promoter activity (FIG. 3F) as well as IL8 mRNA levels (FIG. 3G) in DLD-1^{HIF-kd} but not in DLD-1^{HIF-wt} cells. These observations were confirmed in the Panc-1 pancreatic and PC3 prostate cancer cell lines, indicating the broader importance of KRAS on this alternative regulation of IL-8 (FIG. 9). Furthermore, the stimulatory effect of oncogenic KRAS on NF-κB was observed in hypoxic conditions or in the presence of reactive oxygen species (FIGS. 3D, H). Collectively, these studies indicate that IL-8 can be induced in hypoxia through the activation of NF-κB in the absence of HIF-1, and that oncogenic KRAS can further stimulate NF-κB in hypoxic conditions to up-regulate this alternative angiogenic pathway.

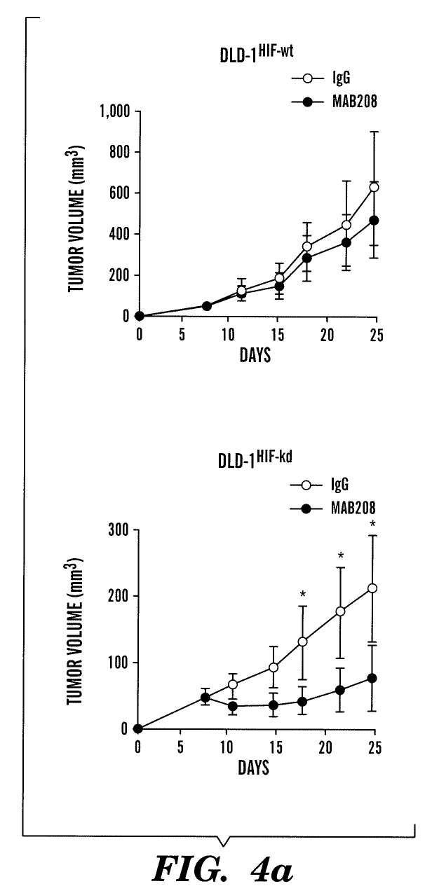

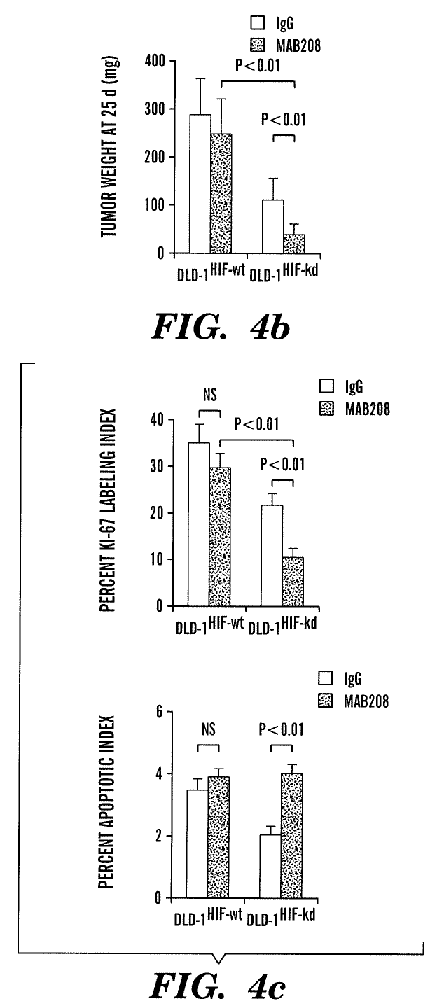

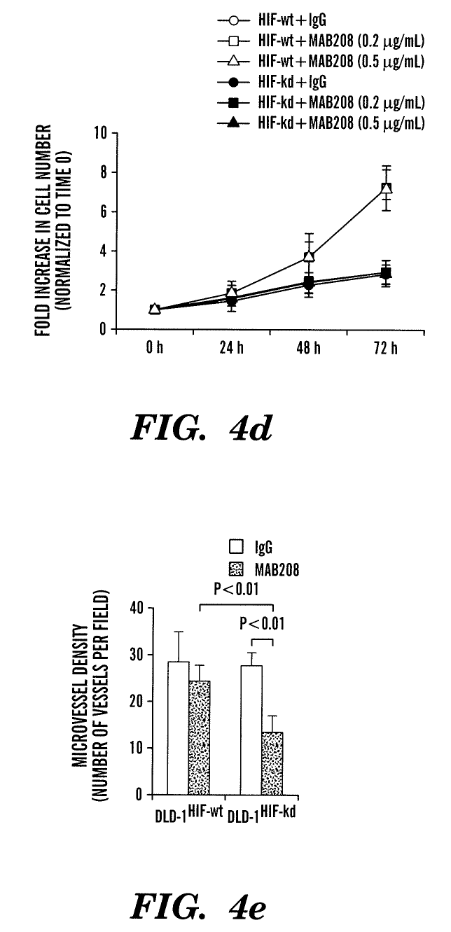

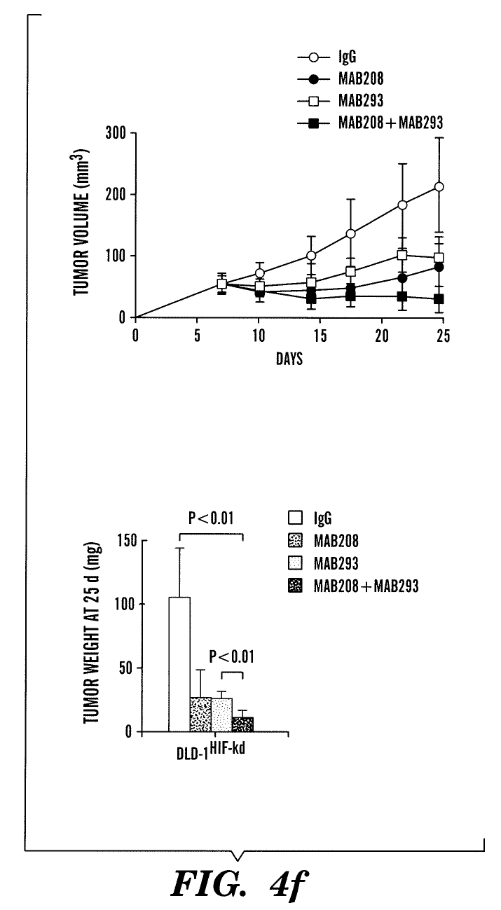

Finally, we sought to determine the functional significance of IL-8 production in HIF-1 deficient tumors. The observation that DLD-1^{HIF-kd} xenografts displayed a marked inflammatory infiltrate (data not shown) was consistent with a functional role for IL-8, a potent neutrophil chemokine¹⁸. Intraperitoneal administration of the IL-8 neutralizing antibody MAB208 resulted in complete regression of 25% of DLD-1^{HIF-kd} xenografts, and among the other detectable DLD-1^{HIF-kd} tumors, there was a 61.3% reduction in tumor volume (P<0.01) and 61.8% reduction in tumor weight (P<0.01) compared to tumors treated with control IgG (Figs. 4A, B). In contrast, there was only a 24.8% (P=0.28) and 15.6% (P=0.35) reduction in tumor volume and weight, respectively, in DLD-1^{HIF-wt} xenografts. Although treatment with MAB208 resulted in a decrease in the Ki-67 labeling index and increase in apoptosis in the DLD-1^{HIF-kd} xenografts (FIG. 4C), in vitro studies revealed that MAB208 did not directly inhibit tumor cell growth (FIG. 4D). Rather, treatment with MAB208 resulted in a dramatic inhibition of angiogenesis. The microvessel density in DLD-1^{HIF-kd} xenografts was reduced 46.5% (P<0.001) compared to a 14.5% reduction (P=0.11) in DLD-1^{HIF-wt} xenografts (FIG. 4E). Confocal microscopy of tumor sections after lectin perfusion verified that vascular integrity was compromised in DLD-1^{HIF-kd} xenografts treated with MAB208 (data not shown). In addition to reduced vessel number, the vessels were markedly narrowed and fragmented. Specifically, the mean vessel diameter fell from 22.4 μm to 5.9 μm (P=0.0002) when DLD-1^{HIF-kd} xenografts were treated with MAB208, but there was no change in the DLD-1^{HIF-wt} xenografts (26.5 μm vs. 24.8 μm with MAB208, P=NS). Neutralization of both IL-8 and VEGF in DLD-1^{HIF-kd} xenografts had an additive effect on the inhibition of tumor growth (FIG. 4F), demonstrating that each factor can regulate tumorigenesis independently.

In summary, we demonstrated that HIF-1α deficiency in colon cancer cells can inhibit proliferation and overall growth but not angiogenesis. There are conflicting reports of the role of HIF-1 in tumor cell proliferation. Hifla^{-/-} ES-derived teratocarcinomas exhibit both reduced as well as increased growth^{2,20}. Among human tumors, overexpression of HIF-1α has been associated with improved survival in patients with head and neck cancers²¹ and HIF-1 can inhibit the growth of renal carcinoma cells²². This may be mediated through the induction of the cell cycle inhibitors p21 and p27²³. It has been speculated that HIF-1 may have intrinsic functions to either promote or inhibit tumor growth that depends upon the cellular context²⁴. The preservation of angiogenesis in our model is explained by persistent expression of VEGF as well as induction of the pro-angiogenic factor, IL-8. IL-8 was stimulated by ROS-mediated activation of NF-κB, and this was enhanced by oncogenic KRAS. Neutralization of IL-8 in HIF-1 deficient tumors led to a dramatic inhibition of angiogenesis and tumor growth. Studies of lung cancer cells harboring a KRAS mutation have also demonstrated a pivotal role for IL-8 in tumor angiogenesis²⁵. Collectively, these findings highlight the complex role of HIF-1α in colorectal tumorigenesis, the diversity of pathways utilized by tumors to stimulate angiogenesis, and the need for combination anti-angiogenic regimens that target both HIF-1 and other targets such as IL-8.

### Example II

### Activation of NF-κB and Preservation of the Angiogenic Response in HIF-1 Deficient Colon Cancer Cells

### Methods

HIF-1α was silenced in the colon cancer cell lines DLD-1 and Caco2 by stable transfection of specific siRNAs. Changes in gene expression patterns induced by hypoxia and HIF-1α silencing were evaluated by cDNA microarray, and results were confined by quantitative real time PCR and ELISA. In vivo effects on angiogenesis were evaluated by inoculating HIF-1 knockdown DLD-1 cells into CD1 nude mice. Regions of intratumoral hypoxia were identified with the Hypoxyprobe reagent, and correlation with vascular endothelial growth factor (VEGF) expression was determined with double immunofluorescence. Microvessel density was measured by CD31 immunohistochemistry.

### Results

In HIF-1α knock-down DLD-1 cells (DLD-1^{HIF-kd}), the hypoxic induction of VEGF was preserved in vitro. When DLD-1^{HIF-kd} cells were grown as xenografts in nude mice, VEGF was also induced, albeit at lower levels. Expression of VEGF correlated with regions of intratumoral hypoxia in HIF-1α knock-down xenografts. Although silencing of HIF-1α impaired tumor growth in vivo, the xenografts remained highly vascularized with microvessel densities that were identical to DLD-1^{HIF-wt} tumors. The pro-angiogenic cytokine interleukin-8 (IL-8) was preferentially induced by hypoxia in DLD-1^{HIF-kd} cells. This induction of IL-8 was mediated by an increased production of reactive oxygen species, resulting in the activation of NF-κB. K-ras, which is commonly mutated in colon cancer, enhanced the production of IL-8 in Caco2^{HIF-kd} and DLD-1^{HIF-kd} cells.

Inhibition of HIF-1 by siRNA does not block tumor angiogenesis in colon cancer. In addition to the persistent induction of VEGF, an alternative angiogenic pathway involving NF-κB and IL-8 was induced when HIF-1α was knocked-down.

### REFERENCES

1. Denko, N. C. et al. Investigating hypoxic tumor physiology through gene expression patterns. Oncogene 22, 5907-5914 (2003).
2. Carmeliet, P. et al. Role of HIF-1 alpha in hypoxia-mediated apoptosis, cell proliferation and tumour angiogenesis. Nature 394, 485-490 (1998).
3. Pugh, C. W. & Ratcliffe, P. J. Regulation of angiogenesis by hypoxia: role of the HIF system. Nat. Med. 9, 677-84 (2003).
4. Tang, N. et al. Loss of HIF-1 alpha in endothelial cells disrupts a hypoxia-driven VEGF autocrine loop necessary for tumorigenesis. Cancer Cell 6, 485-495 (2004).
5. Kung, A. L., Wang, S., Klco, J. M., Kaelin, W. G. & Livingston, D. M. Suppression of tumor growth through disruption of hypoxia-inducible transcription. Nat. Med. 6, 1335-1340 (2000).
6. Semenza, G. L. Targeting HIF-1 for cancer therapy. Nat. Rev. Cancer 3, 721-732 (2003).
7. Hurwitz, H. et al. Bevacizumab plus irinotecan, fluorouracil, and leucovorin for metastatic colorectal cancer. N. Engl. J. Med 350, 2335-2342 (2004).
8. Maxwell, P. H. et al. Hypoxia-inducible factor-1 modulates gene expression in solid tumors and influences both angiogenesis and tumor growth. Proc. Natl. Acad. Sci. U.S.A. 94, 8104-8109 (1997).
9. Ryan, H. E. et al. Hypoxia-inducible factor-1 alpha is a positive factor in solid tumor growth. Cancer Res. 60, 4010-4015 (2000).
10. Mizukami, Y. et al. Hypoxia-inducible factor-1-independent regulation of vascular endothelial growth factor by hypoxia in colon cancer. Cancer Res. 64, 1765-1772 (2004).
11. Pierce, J. W. et al. Novel inhibitors of cytokine-induced IkappaBalpha phosphorylation and endothelial cell adhesion molecule expression show anti-inflammatory effects in vivo. J. Biol. Chem. 272, 21096-21103 (1997).
12. Schreck, R., Rieber, P. & Baeuerle, P. A. Reactive oxygen intermediates as apparently widely used messengers in the activation of the NF-kappa B transcription factor and HIV-1. EMBO J. 10, 2247-2258 (1991).
13. Michiels, C., Minet, E., Mottet, D. & Raes, M. Regulation of gene expression by oxygen: NF-kappaB and HIF-1, two extremes. Free Radic. Biol. Med. 33, 1231-1242 (2002).
14. Chandel, N. S. et al. Mitochondrial reactive oxygen species trigger hypoxia-induced transcription. Proc. Natl. Acad. Sci. U.S.A. 95, 11715-11720 (1998).
15. Chandel, N. S. et al. Reactive oxygen species generated at mitochondrial complex III stabilize hypoxia-inducible factor-1 alpha during hypoxia: a mechanism of O2 sensing. J. Biol. Chem. 275, 25130-25138 (2000).
16. Brand, K. A. & Hermfisse, U. Aerobic glycolysis by proliferating cells: a protective strategy against reactive oxygen species. FASEB J. 11, 388-395 (1997).
17. Seagroves, T. N. et al. Transcription factor HIF-1 is a necessary mediator of the pasteur effect in mammalian cells. Mol. Cell. Biol. 21, 3436-3444 (2001).
18. Sparmann, A. & Bar-Sagi, D. Ras-induced interleukin-8 expression plays a critical role in tumor growth and angiogenesis. Cancer Cell 6, 447-458 (2004).
19. Shirasawa, S., Furuse, M., Yokoyama, N. & Sasazuki, T. Altered growth of human colon cancer cell lines disrupted at activated Ki-ras. Science 260, 85-88 (1993).
20. Ryan, H. E., Lo, J. & Johnson, R. S. HIF-1 alpha is required for solid tumor formation and embryonic vascularization. EMBO J. 17, 3005-3015 (1998).
21. Beasley, N. J. et al. Hypoxia-inducible factors HIF-1 alpha and HIF-2alpha in head and neck cancer: relationship to tumor biology and treatment outcome in surgically resected patients. Cancer Res. 62, 2493-2497 (2002).
22. Raval, R. R. et al. Contrasting properties of hypoxia-inducible factor 1 (HIF-1) and HIF-2 in von Hippel-Lindau-associated renal cell carcinoma. Mol. Cell. Biol. 25, 5675-5686 (2005).
23. Mack, F. A., Patel, J. H., Biju, M. P., Haase, V. H. & Simon, M. C. Decreased growth of Vh1−/− fibrosarcomas is associated with elevated levels of cyclin kinase inhibitors p21 and p27. Mol. Cell. Biol. 25, 4565-4578 (2005).
24. Koshiji, M. & Huang, L. E. Dynamic balancing of the dual nature of HIF-1 alpha for cell survival. Cell Cycle 3, 853-854 (2004).
25. Arenberg, D. A. et al. Inhibition of interleukin-8 reduces tumorigenesis of human non-small cell lung cancer in SCID mice. J. Clin. Invest 97, 2792-2802 (1996).
26. Ofori-Darko, E. et al. An OmpA-like protein from Acinetobacter spp. stimulates gastrin and interleukin-8 promoters. Infect. Immun. 68, 3657-3666 (2000).
27. Khokhlatchev, A. et al. Identification of a novel Ras-regulated proapoptotic pathway. Curr. Biol. 12, 253-265 (2002).
28. Zhang, L., Fogg, D. K. & Waisman, D. M. RNA interference-mediated silencing of the S100A10 gene attenuates plasmin generation and invasiveness of Colo 222 colorectal cancer cells, J. Biol. Chem. 279, 2053-2062 (2004).
29. Belue, G., Smith, L. T. & Tenen, D. G. Use of a promoterless Renilla luciferase vector as an internal control plasmid for transient co-transfection assays of Ras-mediated transcription activation. Biotechniques 26, 24-28 (1999).
30. Mohanty, J. G., Jaffe, J. S., Schulman, E. S. & Raible, D. G. A highly sensitive fluorescent micro-assay of H2O2 release from activated human leukocytes using a dihydroxyplhenioxazine derivative. J. Immunol. Methods 202, 133-141 (1997).
31. C. W. Pugh and P. J. Ratcliffe, Regulation of angiogenesis by hypoxia: role of the HIF system, Nature Medicine, vol. 9, no. 6, pp. 677-684, June 2003.
32. H. Kimura, A. Weisz, T. Ogura, Y. Hitomi, Y. Kurashima, K. Hashimoto, F. D'Acquisto, M. Mqbiuchi, and H. Esumi, Identification of hypoxia-inducible factor 1 ancillary sequence and its function in vascular endothelial growth factor gene induction by hypoxia and nitric oxide, The Journal of Biological Chemistry, vol. 276, no. 3, pp 2292-2298, January 2001.
33. Hurwitz, H. et al. Bevacizumab plus irinotecan, fluorouracil, and leucovorin for metastatic colorectal cancer. N Engl J Med 350, 2335-42 (2004).

All references described herein are incorporated herein by reference.

1. A method of treating undesired angiogenesis in a tissue of a subject having, or at risk of having, an angiogenic disease or disorder by use of a combination therapy comprising i) administering to said subject an inhibitor of hypoxia inducible factor-1 (HIF-1) and ii) administering to said subject a second anti-angiogenic agent wherein the second anti-angiogenic agent is an inhibitor of an angiogenic agent selected from the group consisting of: vascular endothelial growth factor (VEGF), epidermal growth factor (EGF), platelet derived growth factor (PDGF), fibroblast growth factor (FGF), transforming growth factor (TGF), granulocyte colony stimulating factor (GCSF), angiopoietin 1, and interleukin 8 (IL-8).

2. The method of claim 1, wherein said inhibitor of hypoxia inducible factor-1 (HIF-1) and said second agent are administered simultaneously.

3. The method of claim 1, wherein said angiogenic-disease or disorder is cancer.

4. The method of claim 4, wherein said cancer is colon cancer.

5. The method of claim 1, wherein said angiogenic disease is retinopathy of prematurity, diabetic retinopathy or macular degeneration.

6. The method of claim 1, wherein said angiogenic disease or disorder is arthritis or rheumatoid arthritis.

7. The method of claim 1, wherein said angiogenic disease or disorder is psoriasis.

8. A method of treating undesired angiogenesis in a tissue of a subject having, or at risk of having, cancer by use of a combination therapy comprising i) administering to said subject an inhibitor of hypoxia inducible factor-1 (HIF-1) and ii) administering to said subject an inhibitor of interleukin 8 (IL-8), wherein said cancer is selected from the group consisting of colon cancer, pancreatic cancer, lung cancer, and breast cancer.

9. The method of claim 1 or 8, wherein said combination therapy is conducted in conjunction with chemotherapy.

10. The method of claim 1 or 8, wherein said combination therapy is conducted in conjunction with radiation therapy.

11. The method of claim 1 or 8, further comprising administration of a third anti-angiogenic agent.

12. The method of claim 11, wherein the third anti-angiogenic agent is an inhibitor of an angiogenic agent selected from the group consisting of vascular endothelial growth factor (VEGF), epidermal growth factor (EGF), platelet derived growth factor (PDGF), fibroblast growth factor (FGF), transforming growth factor (TGF), granulocyte colony stimulating factor (GCSF), and angiopoietin 1.

13. The method of claim 1 or 8, wherein said administering in steps i) and ii) comprises intravenous, intramuscular, subcutaneous, intradermal, topical, intraperitoneal, intrathecal, intrapleural, intrauterine, rectal, vaginal, intrasynovial, intraocular/periocular, intratumor or parenternal administration.

14. The method of claim 1 or 8, wherein said agents are administered prophylactically.

15. The method of claim 1, wherein said risk for developing an angiogenic disease or disorder is determined genetically or by measuring levels of cancer marker protein.

16. The method of claim 15, wherein the cancer marker protein is selected from the group consisting of; calcitonin, PSA, thymosin β-15, thymosin β-16, and matrix metalloproteinase (MMP).

17. A method of treating undesired angiogenesis in a tissue of a subject having, or at risk of having, an angiogenic disease or disorder by use of a combination therapy comprising i) administering to said subject an inhibitor of hypoxia inducible factor-1 (HIF-1) and ii) administering to said subject a second anti-angiogenic agent wherein the second anti-angiogenic agent is a direct angiogenesis inhibitor selected from the group consisting of: endostatin, angiostatin, thrombospondin and tumstatin.

18. A kit designed for treatment of a subject having, or at risk of having, an angiogenic disease or disorder comprising an inhibitor of hypoxia inducible factor-1 (HIF-1) and at least one other anti-angiogenic agent, wherein the anti-angiogenic agent is selected from the group consisting of: an inhibitor of IL-8, an inhibitor of vascular endothelial growth factor (VEGF), an inhibitor of angiopoietin 1, an inhibitor of epidermal growth factor (EGF), an inhibitor of platelet derived growth factor (PDGF), an inhibitor of fibroblast growth factor (FGF), an inhibitor of transforming growth factor (TGF), and an inhibitor of granulocyte colony stimulating factor (G-CSF).

19. The kit of claim 18, comprising an inhibitor of hypoxia inducible factor-1 (HIF-1) and an inhibitor of IL-8.

20. The kit of claim 18, comprising an inhibitor of hypoxia inducible factor-1 (HIF-1) and an inhibitor of platelet derived growth factor (PDGF).

21. The kit of claim 19, further comprising an inhibitor of vascular endothelial growth factor (VEGF).

22. The kit of claim 19, further comprising an inhibitor of platelet derived growth factor (PDGF).

23. A kit designed for treatment of a subject having, or at risk of having, an angiogenic disease or disorder comprising an inhibitor of hypoxia inducible factor-1 (HIF-1) and at least one other anti-angiogenic agent, wherein the anti-angiogenic agent is selected from the group consisting of endostatin, angiostatin, thrombospondin, and tumstatin.
